(19) United States
(12) Patent Application Publication
(10) Pub. No.: US 2022/0280581 A1
(43) Pub. Date: Sep. 8, 2022

(54) BENEFICIAL BACTERIA AND SECRETORY IMMUNOGLOBULIN A

(71) Applicant: The Regents of the University of California, Oakland, CA (US)

(72) Inventors: Vanessa DUNNE-CASTAGNA, Sacramento, CA (US); David MILLS, Davis, CA (US); J. Bruce GERMAN, Davis, CA (US)

(21) Appl. No.: 17/694,181

(22) Filed: Mar. 14, 2022

Related U.S. Application Data
(63) Continuation of application No. PCT/US2020/052572, filed on Sep. 24, 2020.
(60) Provisional application No. 62/905,260, filed on Sep. 24, 2019.

(51) Int. Cl. A61K 35/745; A61K 47/68; C07K 16/00; A61K 31/702; A23L 33/135; A23L 33/00; A23L 33/18; A23L 33/125; A23K 10/18; A23K 20/163; A23K 20/147

(52) U.S. Cl. CPC A61K 35/745; A61K 47/68; C07K 16/00; A61K 31/702; A23L 33/135; A23L 33/40; A23L 33/18; A23L 33/125; A23K 10/18; A23K 20/163; A23K 20/147; C07K 2317/41; C07K 2317/12; A61K 2035/115

(57) ABSTRACT

The combination of specific immunoglobulins plus activated Bifidobacteria strains or other beneficial bacteria is described with the designed efficacy to colonize unstable microbiome communities in humans or other animals, restoring the keystone Bifidobacteria strains or other beneficial bacteria to compositional and functional importance in the intestine and improve overall health and reduce pathogenic infections in the host. Secretory immunoglobulin A (SIgA), when bound via specific glycans to select commensal bacteria grown on human milk oligosaccharides (HMOs), enhances the colonization potential of commensals through protection from intestinal digestion, enhancing attachment, and dampening host immune response.

### CROSS-REFERENCE TO RELATED APPLICATIONS

This application is a continuation of PCT/US2020/052572, filed Sep. 24, 2020, which claims the benefit of U.S. Provisional Application No. 62/905,260, filed Sep. 24, 2019, each of which is incorporated by reference in its entirety herein for all purposes.

### REFERENCE TO SUBMISSION OF A SEQUENCE LISTING AS A TEXT FILE

The instant application contains a Sequence Listing which has been submitted electronically in ASCII format and is hereby incorporated by reference in its entirety. Said ASCII copy, created on Mar. 8, 2022, is named 081906-1298014-232510US_SL.txt and is 6,644 bytes in size.

### FIELD OF INVENTION

The new establishment or re-establishment of microbiological communities within the anaerobic intestine of humans and animals (microbiome) faces multiple barriers and has proven to be clinically difficult. Disclosed herein are compositions and methods that simultaneously and synergistically overcome those barriers and enable the guided re-establishment of stable and beneficial keystone bacteria within an existing microbial community.

### BACKGROUND

There is considerable trafficking of memory B cells (Antibody or Ig producing cells) throughout intestinal mucosal surfaces represented by a high level of low-affinity, diversified germinal center B cell response to microbial antigens that are required to maintain intestinal homeostasis. The majority of the Ig repertoire produced in the mucosa is somatically hypermutated IgA proteins, with over 25% poly-reactive to microbial associated molecular patterns (MAMPs) and other common antigens [Bunker J J, Erickson S A, Flynn T M, et al.: Natural polyreactive IgA antibodies coat the intestinal microbiota. Science 2017; 358 (6361)].

Mucosal IgA production, through both T cell dependent and independent mechanisms, maintains a very high constant level in the absence of infection, of up to 5 g/kg/day in an adult intestine [Mestecky J, Russell M W, Jackson S, Brown T A: The human IgA system: a reassessment. Clin Immunol Immunopathol 1986; 40 (1):105-14], but can be increased under an inflammatory environment. A T-cell dependent mechanism may be required for preventing dysbiosis and maintaining intestinal homeostasis [Fagarasan S, Muramatsu M, Suzuki K, Nagaoka H, Hiai H, Honjo T: Critical roles of activation-induced cytidine deaminase in the homeostasis of gut flora. Science 2002; 298(5597):1424-7].

SIgA is derived upon transcytosis of dimeric IgA through mucosal epithelial cells bound to the polymeric Ig receptor (pIgR) on the basolateral membrane, and the subsequent apical cleavage of the Ig-bound secretory component (SC), releasing free SIgA into the lumen.

The antigen binding region is the Fab region that may have low or high affinity for specific antigens. SIgA is highly decorated with N-linked glycans-2 sites on each heavy chain totally 8, plus 7 on the secretory component, one on the J-chain, and an additional 2 on the hinge region on IgA2 [Huang J, Guerrero A, Parker E, et al.: Site-Specific Glycosylation of Secretory Immunoglobulin A from Human Colostrum. J Proteome Res 2015; 14 (3):1335-49.]. Glycans play a pivotal role in pathogen clearance, antigen presentation via M cells, and commensal homeostasis. [Perrier C, Sprenger N, cortésy B: Glycans on secretory component participate in innate protection against mucosal pathogens. J Biol Chem 2006; 281 (20):14280-7.]; Dallas S D, Rolfe R D: Binding of Clostridium difficile toxin A to human milk secretory component. J Med Microbiol 1998; 47 (10):879-88].

Not only are these glycans on SIgA necessary for mucosal response to pathogens, but more recent studies have shown utility of these glycans in binding to commensal bacteria and promoting immune tolerance and maintaining homeostatic regulation. [Mathias A, Corthésy B: Recognition of Gram-positive Intestinal Bacteria by Hybridoma- and Colostrum-derived Secretory Immunoglobulin A Is Mediated by Carbohydrates. J Biol Chem 2011; 286 (19): 17239-47]. Immune Exclusion

SIgA may act through agglutination and neutralization, excluding pathogens from binding to epithelial cells via Fab-dependent, high affinity interactions to pathogen surface antigens. Bacterial pathogens may be coated with SIgA forming immune complexes that can prevent bacterial adhesion to epithelial cells and reduce pathology or may alter membrane integrity potentially through cross-linking of several O-antigens [Moor K, Diard M, Sellin M E, et al.: High-avidity IgA protects the intestine by enchaining growing bacteria. Nature 2017; 544(7650:498-502]. [Forbes S J, Martinelli D, Hsieh C, et al.: Association of a protective monoclonal IgA with the O antigen of Salmonella enterica serovar Typhimurium impacts type 3 secretion and outer membrane integrity. Infect Immun 2012; 80 (7):2454-63].

The gut of the neonate contains an underdeveloped gut-associated lymphoid tissue (GALT) and a naïve adaptive immunity which can take up to ten days to become stimulated [Gibbins H L, Proctor G B, Yakubov GE, et al.: SIgA Binding to Mucosal Surfaces Is Mediated by Mucin-Mucin Interactions. PLOS ONE 2015; 10 (3):e0119677]. These conditions-can lead to an imbalanced inflammatory response without proper guidance from the mother's passive immune defenses in breast milk that aid in establishing a regulated environment during the initial onslaught of microbial colonization. SIgA is the primary mucosal antibody found in the highest abundance over other immunoglobulins in milk, with concentrations up to 15 mg/ml in colostrum and ˜1 mg/ml in mature milk [Torow N, Marsland B J, Hornef M W, et al.: Neonatal mucosal immunology. Mucosal Immunol 2017; 10 (1):5-17], providing the nursing infant 0.5 — 1 g/day.

cortésy et al. demonstrated that in the respiratory tract, tissue localization was dependent upon the glycosylation of the SIgA molecule [cortésy B, Phalipon A.: Molecular definition of the role of secretory component in secretory IgA-mediated protection at mucosal surfaces. Journal of Allergy and Clinical Immunology 2002; 109 (1, Supplement 1):5113]. Upon intranasal challenge with Shigella flexneri, the glycosylated SIgA molecule was viewed in close association with the mucosa, and dissemination of the pathogen was confined to the nasal cavity, but deglycosylation of SIgA led to pathogenic colonization in the deep lung alveoli. While research supports the association of glycosylated SIgA with the outer mucosal layer in the large intestine, Rogier et al. showed that the mucin MUC2 protein in the innermost mucosal lining of the gut epithelium is important not in binding to SIgA, but in excluding this complex from interacting with the epithelia [Rogier E W, Frantz A L, Bruno M E C, Kaetzel C S: Secretory IgA is Concentrated in the Outer Layer of Colonic Mucus along with Gut Bacteria. Pathogens 2014; 3 (2):390-403].

### Milk SIgA and Infant Commensal Colonization

Whereas the protective role of pathogen-targeted secretory milk antibodies is well established, recent studies have emerged focusing on the functional consequences of milk SIgA association with commensals in the development of the newborn microbiota. Studies utilizing BugFACS reveal distinct microbiota coated with SIgA under homeostatic conditions or in malnourished Malawian infants [Kau A L, Planer J D, Liu J, et al.: Functional characterization of IgA-targeted bacterial taxa from undernourished Malawian children that produce diet-dependent enteropathy. Sci Transl Med 2015; 7 (276):276ra24; Planer J D, Peng Y, Kau A L, et al.: Development of the gut microbiota and mucosal IgA responses in twins and gnotobiotic mice. Nature 2016; 534 (7606):263-6.]Malnourished infants had a higher relative abundance of Enterobacteriaceae associated with intestinal inflammation, which represented a significant portion of the SIgA-associated taxa. Conversely, healthy infants showed consistently high SIgA-association in members of the genera Akkermansia and Clostridium, regardless of dietary intake or age (1-24 mo).

NEC development has been correlated with a loss of IgA association to Enterobacteriaceae, through unclear mechanisms.

The art would suggest that any commensal organism can bind with any SIgA to form an complex (U.S. Pat. Nos. 10,501,530, 9,173,937 and 9,629,908). However, the invention disclosed herein provides methods and mechanisms that select for effective combinations requiring bacteria specific and SIgA features including glycan mediated differences in binding.

### SUMMARY OF INVENTION

The herein disclosed invention describes a method of stimulating beneficial bacteria in a gut of an individual, the method comprising, administering beneficial bacteria and an immunoglobulin to the individual. Compositions and methods that administer immunoglobulin and beneficial bacteria may additionally be present in an immunoglobulin-beneficial bacteria complex. Such complexes may be used to protect the bacteria from gastric digestion and deliver a complex to modulate the microbiome, prevent or treat a disease or condition. In particular, stimulating the persistence and viability of Bifidobacterium in a gut of an individual, the method comprising, administering Bifidobacterium and an immunoglobulin to the individual. Compositions and methods that administer immunoglobulin and Bifidobacterium may additionally be present in an immunoglobulin-Bifidobacterium complex. Such complexes may comprise immunoglobulin fragments. Reference in this disclosure to such complexes shall be understood to mean whole immunoglobulins and/or immunoglobulin fragments forming a complex with a Bifidobacterium.

The complexes described herein may be administered to an individual. The individual may be a human or a non-human mammal. The non-human mammal may include, but is not limited to, a pig, cow, horse, dog, cat, camel, rat, mouse, goat, sheep, or water buffalo. The non-human mammal may also include goat, sheep, water buffalo, camel or others whose milk may be consumed by humans. The non-human mammal may be an animal used in food production, a performance animal, or a domesticated pet. Any of the above may be a newborn, weaning, adult or geriatric animal. The human individual may be an infant, a preterm or premature infant who may be born with a gestational age of less than 33 weeks, the preterm babies may be a very low birth weight (VLBW), or low birth weight (LBW), a term infant (0-3 months), an infant 3-6 months, an infant (6-12 months), a weaning infant (4-12 months), a weaned infant (12 months to 2 years) and child (1-16 years), an adult (16-70 yr), or an older adult (70-100+yr). The preterm infant may be at risk of developing necrotizing enterocolitis (NEC). The infant or child may be at increased risk for diarrheal diseases.

A composition comprising the beneficial bacteria and immunoglobulin may be administered as a food product or a pharmaceutical composition. The beneficial bacteria and immunoglobulin may be delivered contemporaneously as a pre-formed complex, or as components that can self-assemble prior to administration or post-administration (i.e., following consumption).

In some embodiments, the food product is selected from the group consisting of infant formula, follow-on formula, toddler's beverage, milk, soy milk, fermented milk, fruit juice, fruit-based drinks, meal replacer, and sports drink. In other embodiments, the food product may be a liquid or powdered human milk product including, but not limited to human milk fortifiers (bovine or human), processed donor milk, or milk fractions. In some embodiments, the food product is maintained in a dried state and may be added to a liquid at time of consumption by the individual. In other embodiments, the product is formulated to be stable in a liquid form. The liquid form may be an aqueous liquid or it may be an anhydrous liquid such as an oil. The oil may be a solid or liquid at room temperature. In some embodiments the food product may take the form of a powder.

In some embodiments the pharmaceutical composition may take the form of a pill, tablet, sachet, powder, or oil suspension.

In any of the embodiments whether it is pharmaceutical composition or food product, a beneficial or keystone bacteria may be of the genera Bifidobacterium and/or Lactobacillus. The Bifidobacterium may be selected from the group consisting of B. longum subsp. infantis, B. longum subsp. longum, B. pseudocatenulatum, B. bifidum, B. kashiwanohense, B. adolescentis, and B. breve. The Lactobacillus may be selected from the group consisting of L. acidophilus, L. rhamnosus, L. casei, L. paracasei, L. plantarum and L. reuteri. In a preferred embodiment, the Bifidobacterium is B. infantis or B. pseudocatenulatum. In a most preferred embodiment, the Bifidobacterium is B. infantis.

In some embodiments of this invention the B. infantis may be activated. In some embodiments of this invention the HMO-activating agent is selected from at least one of the group lacto-N-biose (LNB), N-acetyl lactosamine, lacto-N-triose, lacto-N-tetraose (LNT), lacto-N-neotetraose (LNnT), fucosyllactose (FL), lacto-N-fucopentaose (LNFP), lactodifucotetraose, (LDFT) sialyllactose (SL), disialyllacto-N-tetraose (DSLNT), 2′-fucosyllactose (2FL), 3′-sialyllactosamine (3SLN), 3′-fucosyllactose (3FL), 3′-sialyl-3-fucosyllactose (3S3FL), 3′-sialyllactose (3SL), 6′-sialyllactosamine (6SLN), 6′-sialyllactose (6SL), difucosyllactose (DFL), lacto-N-fucopentaose I (LNFPI), lacto-N-fucopentaose II (LNFPII), lacto-N-fucopentaose III (LNFPIII), lacto-N-fucopentaose V (LNFPV), sialyllacto-N-tetraose (SLNT), their derivatives.

In any of the embodiments, the SIgA is not from the mother of the individual in need. It may be from a milk source or may be recombinant or synthetic.

An immunoglobulin is selected from the group consisting of secretory immunoglobulin A (SIgA), IgA, IgM, IgG, IgE, IgD or fragments thereof. Such fragments may comprise at least 20, 40, 60, 80, or 100 amino acids. In some embodiments, the Ig fragments are fragments of Ig glycoproteins or glycopeptides. Any mention of “immunoglobulin” in this disclosure shall be understood to refer to an immunoglobulin and/or any fragment thereof.

Immunoglobulins referenced herein may be recombinant or otherwise from a synthetic source. Further, immunoglobulins as herein referenced may be from a heterogenous milk-derived immunoglobulin fraction. In some embodiments the engineered immunoglobulin may be selected based on its binding affinity. Such binding affinity of the immunoglobulin active site may be targeted to Bifidobacterium to aid in the formation of an immunoglobulin-Bifidobacterium complex. Such binding affinity of the immunoglobulin active site may alternatively be selected to target enteric pathogens, or any other non-Bifidobacterium species in order to assist in mitigation of the growth of such other species. Immunoglobulins referenced herein may additionally be selected for the ability of the glycan portion of the immunoglobulin to bind to the surface of Bifidobacterium in such a way as to aid in the formation of immunoglobulin-Bifidobacterium complexes. In some embodiments a mixture of immunoglobulins expressing a variety of active sites, which may target a plurality of organisms, may be selected and utilized according to the needs of the user. Such a mixture may include immunoglobulins targeted at Bifidobacteria in addition to others targeted at non-Bifidobacterium.

Any embodiment may comprise a pharmaceutical composition or food product comprising beneficial bacteria and immunoglobulin in a dose sufficient to enhance colonization of the beneficial bacteria compared to administration of the beneficial bacteria alone.

Any of the embodiments may further comprise administering one or more oligosaccharides and/or polysaccharides to the individual in a sufficient amount to enhance colonization of the gut by the beneficial bacteria compared to not administering the oligosaccharide or polysaccharide. In some embodiments, the oligosaccharide is a human milk oligosaccharide (HMO). In some embodiments the HMO may be selected from HMO-activating agent is selected from at least one of the group lacto-N-biose (LNB), N-acetyl lactosamine, lacto-N-triose, lacto-N-tetraose (LNT), lacto-N-neotetraose (LNnT), fucosyllactose (FL), lacto-N-fucopentaose (LNFP), lactodifucotetraose, (LDFT) sialyllactose (SL), disialyllacto-N-tetraose (DSLNT), 2′-fucosyllactose (2FL), 3′-sialyllactosamine (3SLN), 3′-fucosyllactose (3FL), 3′-sialyl-3-fucosyllactose(3S3FL), 3′-sialyllactose (3SL), 6′-sialyllactosamine (6SLN), 6′-sialyllactose (6SL), difucosyllactose (DFL), lacto-N-fucopentaose I (LNFPI), lacto-N-fucopentaose II (LNFPII), lacto-N-fucopentaose III (LNFPIII), lacto-N-fucopentaose V (LNFPV), sialyllacto-N-tetraose (SLNT), or their derivatives.

Some embodiments of this invention include a pharmaceutical composition or food product comprising Bifidobacterium and immunoglobulin in a dose sufficient to enhance colonization of the Bifidobacterium compared to administration of Bifidobacterium alone.

Any embodiment of this invention may be used to prevent or to treat an enteric disease. In any embodiment of this invention such disease may be an auto-immune disease. In any embodiment of this invention such disease may be a dysbiosis-associated disease. In any embodiment of this invention such disease may be an infection of an enteric pathogen.

### DESCRIPTION OF FIGURES

FIG. 1. SIgA association to commensals is concentration dependentₛ and reaches saturation at or below concentrations found in breastmilk (A), as determined by flow cytometry (n=30). Aggregate formation increases upon increased association to SIgA in LR (B). Bacteria varied in percent association (C), with some showing very poor association at the highest concentration tested (1000 μg/1e7 CFU). Bacteria increased association to SIgA when first grown on 2′FL (BLI, p=0.0014, BP, p=0.056, and BLL, p=0.38) (D).

FIG. 2. SIgA increases viability post-digestion in vitro. When grown on glucose, BLI shows 1e5 CFU (sd 5e5 CFU) viable bacteria post-digestion when no SIgA is provided, and viability increases to 8.9e5 CFU (sd 3.3e5 CFU) when 1000 μg SIgA/1 e7 CFU is provided (p=0.002). A regression plot of BLI viability with SIgA association (B) shows a slope of 8.2e4 CFU per % population SIgA association (R²=0.53, p=0.0074). A live-dead flow cytometry analysis of BLL (C) shows increased viable bacteria from 29.05% live (sd 6.6%) to 50.85% live (sd 0.92%) after 1000 μg SIgA/1e7 CFU is provided (p=0.04).

FIG. 3. SIgA increases viability post-digestion in vitro. When grown on glucose, B. infantis shows 1e5 CFU (sd 5e5 CFU) viable bacteria post-digestion when no SIgA is provided, and viability increases to 8.9e5 CFU (sd 3.3e5 CFU) when 1000 μg SIgA/1e7 CFU is provided (p=0.002) (B), and a regression plot shows association between percent SIgA association and increased viability (R²=0.785, p=0.0001) where the p-value indicates that the slope is significantly different from zero (A). When grown on LNnT, B. infantis shows 1.2e4 viable CFU (sd 4.2e3 CFU) post-digestion when no SIgA is added, and 1.3e5 CFU (sd 7.4e4) after 1000 μg SIgA/1e7 CFU is provided (p=0.0243) (D). A regression plot shows a slope of 1.2e3 CFU per % population SIgA association (R²=0.62, p=0.0025) (C). B. pseudocatenulatum regression plot shows a slope of 8.2e4 CFU per % population SIgA association (R²=0.53, p=0.0074) (E). Live-dead analysis of B. breve shows no viable bacteria when no SIgA is added, but 57.8% (sd 4.5%) viable bacteria after 1000 μg SIgA/1e7 CFU is provided (p=0.0051). Both Lactobacillus species L. acidophilus (G) and L. reuteri (H), when grown on glucose, show a negative association to SIgA post-digestion (m=−8.9e3, R²=0.2, p=0.15 for L. acidophilus, and m=−3.9e4 CFU/% SIgA association, R²=0.044, p=0.59 for L. reuteri).

FIG. 4. SIgA improves adherence of commensals to colonocyte co-cultures. BLI shows increased adherence to the co-culture with the addition of SIgA when first grown on glucose (3.5e6 CFU increased adherence, p=0.0168) (A) or LNnT (7.5e5 CFU, p=0.0164) (B), and BLL showed increased adherence to colonocytes with SIgA association only when first grown on 2′FL (4e3 increase, p=0.024) (C). A regression plot of lactose-grown LR (D) had increased adherent bacteria with increased association with SIgA (slope=37.7). BLI (E) showed similar correlation when grown on lactose (slope=4.6e3). Slope units are adherent CFU per % population associated with SIgA, and p value on regression plots indicate if the slope is not zero.

FIG. 5. Heat map of barrier function and immune gene expression changes to colonocytes when BLI only (0), or BLI complexed to SIgA (1000, 1000 μg SIgA per 1e7 CFU.

FIG. 6. SIgA association with BLI is concentration dependent and stable over time

FIG. 7. Sal4 association to both strains of Salmonella was concentration-dependent. (A) and although at 5 μg Sal4 per 1×10⁷ CFU both strains has similar association to Sal4, at 30′ g Sal4 there was higher association of the wild-type JS107 than the mutant SJF10 to the antibody (p=). Sal4 prevented invasion of both the wild-type (white columns) and the mutant (blue columns) into colonocytes (B) when pre-incubated (ST-Sal4), but when the BLI-Sal4 complex was added to the colonocytes prior to ST challenge, only the wild-type was prevented from invasion (BLI-Sal4 ST). A competitive index calculation (C) of all invasion assays (n=9 from three separate trials) shows a selective reduction of the wild-type strain both when Sal4 was added directly to the ST mix prior to colonocyte challenge (ST-Sal4) or when BLI-Sal4 complex was added first to the colonocytes prior to ST challenge (BLI-Sal4 ST).

FIG. 8. Gene expression changes in colonocytes as compared to PBS control. (A) A heatmap of barrier function genes including MUCSAC (mucin protein produced by HT-29 cells), MUC13 (mucin protein produced by Caco2 cells), Claudin 1, Occludin and Junction Adhesion Molecule (JAM), and immune function genes including interleukin 8 (IL8), lysozyme, polymeric Ig receptor (pIgR), and Receptor Interacting Protein Kinase 1 (RIPK1). (B) IL-8 gene expression changes with various treatments over PBS-treated colonocytes.

FIG. 9. Brightfield microscopy of a Gram stain of various combinations of BLI, Sal4 and ST. (A) BLI with no Sal4 has natural clustering, but is increased in aggregation when 200 μg per 1×10⁷ CFU was added (D). ST shows no aggregate formation without Sal4 (B) but has a high degree of aggregation with 30 μg Sal4 per 1×10⁷ CFU (E). BLI and ST together show little association without Sal4 (C), but have significant BLI-ST clustering when BLI is first pre-incubated with Sal4 for 30 m followed by the addition of ST (F).

FIG. 10. Schematic (A) showing the experimental design for the mouse trials. BI, BI-Sal4 complex, or PBS was provided via oral-gastric gavage to 7 week-old female BALBc mice for three days, followed by ST challenge on either d5 or d7. Mice were provided 10% 2′FL in their drinking water for the trial. Competitive index (B) shows the ratio of wild-type JS107 to mutant SJF10 strains collected from the Peyer's patches of mice during necropsy. BI-Sa14 complex reduced wild-type JS107 by roughly 30% over mutant SJF10 when ST was challenged 3 days after oral administration of the probiotic complex (d5), but there was no effect when ST was challenged two days later (d7). Student t-test of the CI of each treatment vs untreated ST control (ST d7) revealed statistical significance of both ST-Sal4 positive control (CI=0.25, p=0.0001) and BI-Sal41 STd5 (CI=0.64, p<0.01).

FIG. 11. (A) BLI persistence as detected by CFU/g feces in BALBc mice 1 day (d4), 3 days (d6) and 5 days (d8) post-oral administration. (B) Persistence data only for 5 days post-gavage (d8). Treatment groups were as follows: A: BLI only with no Sal4 and mice provided water. B: BLI with no Sal4 and mice provided 10% 2′FL. C: BLI pre-incubated with 100 μg per 1e7 CFU, and mice provided water. D: BLI pre-incubated with 100 μig per 1e7 CFU, and mice provided 2′FL. Data shows that 2′FL alone is sufficient to improve persistence (A to B), that Sal4 alone can improve persistence (A to C), and that there is a combination effect (D) of both Sal4 and 2′FL.

FIG. 12. When pre-incubated with milk SIgA, B. infantis is found 10× higher concentration in the feces of BALBc mice one day post oral administration, indicating improved protection from digestion.

### DETAILED DESCRIPTION OF THE INVENTION

This disclosure describes use of milk-derived or recombinant SIgA (or other immunoglobulins) to introduce HMO-grown or “activated” commensal Bifidobacterium species into the mammalian gastrointestinal tract. Examples include populations susceptible to mortality associated with enteropathogenic infections, including term infants and children at risk or suffering from diarrheal diseases (for example in developing regions), and preterm infants who face the risk of necrotizing enterocolitis, but may also be used in other populations and age groups. For example, but not limited to, those that travel to regions known for higher risk of enteric infections.

Modification of complex microbial communities has been challenging, as most probiotics provided orally do not colonize for any significant length of time except in breast-fed infants who receive an HMO-consuming commensal that can establish a unique ecological niche in the HMO-consuming infant gut. The utility of the proposed methods in modifying an established microbiota without the requirement for concurrent HMO supplementation provides wide application of the product.

The microbiome or microbial communities that make up the gastrointestinal tract or gut of different host mammals have specific species that play ecological roles, but may also be susceptible to invasion by pathogens (enteropathogens) or opportunistic pathogens. Keystone species or beneficial bacteria within the gut means commensal bacteria occupying a stable, abundant and functional role within the community.

Human milk oligosaccharides or HMO are a fraction of human milk known to be largely undigestible to the infant consuming them, but instead may feed certain bacterial species within the intestinal microbiome. Oligosaccharide structures of interest may be enriched or processed from mammalian milks, such as bovine or goat. Alternatively oligosaccharide can be of enzymatic or synthetic origin. Oligosaccharides are typically 3-20 sugar residues or moieties, but may preferentially be 3-8 residues. HMOs are exemplified by structures such as but not limited to lacto-N-biose (LNB), N-acetyl lactosamine, lacto-N-triose, lacto-N-tetraose (LNT), lacto-N-neotetraose (LNnT), fucosyllactose (FL), lacto-N-fucopentaose (LNFP), lactodifucotetraose, (LDFT) sialyllactose (SL), disialyllacto-N-tetraose (DSLNT), 2′-fucosyllactose (2FL), 3′-sialyllactosamine (3SLN), 3′-fucosyllactose (3FL), 3′-sialyl-3-fucosyllactose(3S3FL), 3′-sialyllactose (3SL), 6′-sialyllactosamine (6SLN), 6′-sialyllactose (6SL), difucosyllactose (DFL), lacto-N-fucopentaose I (LNFPI), lacto-N-fucopentaose II (LNFPII), lacto-N-fucopentaose III (LNFPIII), lacto-N-fucopentaose V (LNFPV), sialyllacto-N-tetraose (SLNT), their derivatives, or combinations thereof.

Polysaccharides are dietary fractions of greater than 20 residues and may be typically much longer that reach the large intestine or colon that may be cleaved into oligosaccharides or used as fermentation substrates for certain bacterial species in the microbiome.

Dysbiosis for the purpose of this invention means an absence or insufficiency of one or more keystone species and/or the presence or overabundance of one or more enteropathogens.

Immunoglobulin fragment means an incomplete immunoglobulin structure that has at least 10, 20, 40, 80 and at least 100 amino acid residues. In particular, fragments suitable for the invention are ones that are glycoproteins or glycopeptides that anchor select bacteria or those that are activated to increase binding efficiency to the immunoglobulins through a glycan mediated interaction. The fragment may contain some or all of the Ig-bound secretory component (SC) needed to release free SIgA. A glycosylated SC region alone may be used to coat a bacteria in glycosylated protein. In some embodiments, the antigen presenting region may have low or high affinity, which is the strength of the interaction between the immunoglobulin antigen-binding cleft and its concordant antigen, with a dissociation constant (K_{D}) 10⁻⁴ or less, or that may have high or low avidity, which is the combined strength of the interaction between the immunoglobulin and its concordant antigen based on affinity, valency and ligand availability. In some embodiments, the SIgA is engineered (may also be referred to as recombinant or synthetic) to deliver stable glycan mediated binding to a keystone organism with epitopes against enteropathogens known to be involved in NEC or may be organisms known to cause childhood diarrheal diseases globally.

Immunoglobulin-commensal organism complex. The formation of a complex between SIgA and a bacteria may be a mechanism to protect the bacteria during gastrointestinal tract (GI) transit through the stomach (low pH and protease rich environment) to the large intestine or colon where the largest microbial communities reside. These microbial communities are known to colonize or persist in this anaerobic environment and provide functional benefit to the host. The sIgA component may encapsulate or provide a protein coat around the bacteria.

Complexes used in this invention may be pre-formed prior to consumption by an individual in need of the complexes containing beneficial keystone species and SIgA directed against one or more enteropathogens. Alternatively, the SIgA fragment contains the Ig-bound secretory component (SC) region but not an antigen binding region and acts solely to encapsulate the keystone or beneficial bacteria to improve survival during product storage, transit through the gastrointestinal tract and/or the persistence, stability or colonization in the microbial community. The food or pharmaceutical composition may have the complexes pre-formed during the manufacturing process that may or may not be added to liquid before consumption or may be in a tablet format. Alternatively, the protocol or treatment regime for introducing complexes to prevent infection, reduce dysbiosis or treat a known infection may involve mixing of a dry powder containing part of the complex while the other part of the complex is in a liquid composition. In some embodiments, a desired reaction time is used to mix the 2 parts contemporaneously to create the complex in the time prior to consumption by the individual. Alternatively, they are not pre-assembled.

For the purposes of this invention an effective pool or cocktail of SIgA may refer to either a food or therapeutic composition in which the antigen binding region or one or more sIgA are directed against enteropathogens that are the cause of dysbiosis, infection, or other intestinal distress. Intestinal distress is taken to mean symptoms, such as diarrhea, constipation, intestinal cramps, colitis or diaper rash that may be caused for example by travel, stress or antibiotics or dysbiosis. An effective pool or cocktail may also mean an SIgA linked to a commensal or a keystone bacteria that is considered beneficial. Beneficial is defined as having a benefit to the microbiome or microbial community and/or the host.

### Compositions of Immunoglobulins

Immunoglobulin may be selected from the group comprising one or more of secretory immunoglobulin A (SIgA), dimeric IgA (dIgA), monomeric IgA, secretory IgM (SIgM), IgM, IgG, IgE, IgD or fragments thereof. The immunoglobulin fragment may comprise at least 10, 20, 40, 60, 80, or at least 100 amino acids of the immunoglobulin. The immunoglobulin or immunoglobulin fragment may contain one or more glycosylated protein components. They may be N or O linked glycans with high mannose, complex, or hybrid arrangements that may include residues of mannose, glucose, galactose, fucose, sialic acid, and N-acetylglucosamine.

Any immunoglobulin regardless of how it is derived (natural or recombinant) may be used as a component of a composition intended to be delivered to the intestine of a subject in need of keystone bacteria. In some embodiments, a heterogeneous pool of processed human milk SIgA may be delivered as part of compositions described herein. Human milk may be processed to enrich, partially purify or otherwise be processed to yield a stable source of human milk SIgA for administration to a subject in need. The processing of human milk yields human milk products that differ from the natural state and may be enriched or missing key components that would naturally provide complete nutrition to an infant. The human milk products may also contain one or more HMO including but not limited to 2′FL, LNT or LNnT. The composition comprising a heterogeneous pool of SIgA may be in a liquid or powdered form Other mammalian milks may be processed to generate a heterogeneous pool of sIgA against a targeted set of enteropathogens. In some embodiments, a mammalian system such as, but not limited to a cow, goat is treated to deliver humanized sIgA of other immunoglobulins. A heterogeneous pool is any composition that contains epitoped against more than one antigen that may be for a one or more enteropathogens.

In other embodiments, a recombinant monoclonal SIgA (rSIgA) derived from a mammalian source with similar efficacy may be used. In some embodiments, non-mammalian systems for sIgA production are used provided they deliver a glycosylated immunoglobulin protein. In some embodiments, a highly specific rSIgA cocktail selective against key enteropathogens prevalent in a geographical region are made from a recombinant system such as, but not limited to mammalian cell lines, or other systems known in the art that are capable of producing antibodies that may or may not have glycosylation. The glycosylation may be humanized or may be engineered to increase binding efficiency to the commensal organism.

This should lead to the generation of highly specific rSIgA cocktail selective against key enteropathogens prevalent in a geographical region, that could be administered to susceptible individuals and serve to protect against invading pathogens. Immunoglobulins may be effective against, such enteric pathogens or toxins of viral, fungal or bacterial origin causing diarrheal diseases such as but not limited to rotavirus, Salmonella, Shigella, Camplyobacter, Cryptosporidium, or Escherichia coli or other problematic organisms such as but not limited to Clostridium difficile. In some embodiments, a recombinant SIgA can target an epitope for a particular antigen, such as an enterotoxin, a surface protein, such as those involved in adhesion or invasion of the organism. One skilled in the art would look to develop a recombinant sIgA with an epitope that reacts in the first instance to neutralize a toxin, such as, but not limited to the following enterotoxins, cytotoxins or exotoxins: Clostridium enterotoxin from Clostridium perfringens, Cholera toxin from Vibrio cholerae, Staphylococcus enterotoxin B from Staphylococcus aureus, Shiga toxin from Shigella dysenteriae, or those from Bacillus cereus, or Toxin A or B from Clostridium difficile.

In some embodiment, the immunoglobulin concentration may be calculated as milligrams/milliliter (mg/ml) micrograms μg/g of the final composition of either a liquid or powder composition. The final concentration of Immunoglobulin may be less than 0.5 grams per day, may be between 0.5-1 gram/day, 1-5 grams/day, 5-10 grams/day or greater than 10 grams/day. Alternatively, concentration may be calculated in mg/ml. Ranges may include 0.1 mg/ml-50 mg/ml. It may also be calculated as grams per kilogram body weight per day. For example, a composition my deliver at least 0.05 — 5 grams/Kg body weight per day, greater than 0.1, 1, 5, 10, 15, 20 grams/kg body weight/day.

### Compositions of Bacteria

Keystone or beneficial bacteria are exemplified by species selected from the genus of Bifidobacterium or Lactobacillus. However, one skilled in the art would understand, that the selection criteria developed here, may be used to test the binding and benefit of forming complexes with other genus of commensal organisms. Bifidobacterium may be selected from the group consisting of, but not limited to B. infantis, B. longum, B. pseudocatenulatum, B. Bifidum or B. breve. The Lactobacillus may be selected from the group consisting of, but not limited to L. acidophilus, L. rhamnosus, L. casei, L. paracasei, L. plantarum and L. reuteri.

The commensal organism or probiotic bacteria may be administered to deliver a daily intake reported by colony forming units (CFU) delivered or consumed. The daily intake of 1 million CFU/gram of composition through 100 billion CFU/gram of composition is calculated as part of the diet. The CFUs may be delivered in a single serving or multiple servings per day. In preferred embodiments, the daily intake is at least 100 million, at least 300 million, at least 1 billion, at least 4 billion, at least 6 billion, at least 8 billion, at least 13 billion, or at least 18 billion CFU/gram of composition.

HMO-grown or “activated” means a bacteria grown with HMO to change gene expression and cell surface markers. In some embodiments, bacteria may be fermented with one or more HMO or HMO like molecules to form an activated bacteria prior to administration. Activation includes fermentation with HMO as a carbon source, such as LNT, LNnT, or 2′FL through the exponential growth. The cell surface expression changes from that grown on glucose or lactose rending the bacterial cells more adherent to the immunoglobulin. Activation of B. infantis for example may be activated using a method described in USP 10, 716,816 that can include various combinations of mammalian milk oligosaccharides. The bacteria activated during fermentation may be harvested and lyophilized for use with Immunoglobulins. Embodiments may involve the powdered (dried or lyophilized) bacteria being dry blended with SIgA. In some embodiments, the activated bacteria slurry or cell suspension in liquid form are mixed at specific ratios of CFU/ml with μg sIgA protect the bacteria during the lyophilization process and/or storage. SIgA/bacteria ratios may be 1-5000 μg sIgA to 10⁴ to 10¹² CFU/ml.

Oligosaccharides may be used as other components in the composition delivered to the intestine of the individual used. The oligosaccharide may be used to maintain activation in vivo or otherwise support the persistence or colonization, viability or effectiveness of the keystone species in a microbial community Exemplary oligosaccharides include but are not limited to one or more of lacto-N-biose (LNB), N-acetyl lactosamine, lacto-N-triose, lacto-N-tetraose (LNT), lacto-N-neotetraose (LNnT), fucosyllactose (FL), lacto-N-fucopentaose (LNFP), lactodifucotetraose, (LDFT) sialyllactose (SL), disialyllacto-N-tetraose (DSLNT), 2′-fucosyllactose (2FL), 3′-sialyllactosamine (3SLN), 3′-fucosyllactose (3FL), 3′-sialyl-3-fucosyllactose (3S3FL), 3′-sialyllactose (3SL), 6′-sialyllactosamine (6SLN), 6′-sialyllactose (6SL), difucosyllactose (DFL), lacto-N-fucopentaose I (LNFPI), lacto-N-fucopentaose II (LNFPII), lacto-N-fucopentaose III (LNFPIII), lacto-N-fucopentaose V (LNFPV), sialyllacto-N-tetraose (SLNT), their derivatives, or combinations thereof.

Polysaccharides that may be include mucin or mucin fragments from animal sources or may be from dietary plant sources.

### Formulations and Applications of the Compositions

In some embodiments, the bacteria-SIgA combination is selected based on its ability to survive gastric digestion or improve colonization above what is possible for the bacteria alone in an established microbiome.

In some embodiments, the beneficial bacteria and immunoglobulin are components of a food product or in other embodiments a pharmaceutical composition.

The food product may be selected from the group consisting of human milk products including but not limited to human milk fortifier (bovine or human), processed donor milk, preterm infant formula, term infant formula, follow-on formula, toddler's beverage, milk, soy milk, fermented milk, fruit juice, fruit-based drinks, and sports drink. The infant formula may be a ready to drink formula or one that is powdered to which water is added.

The food product or pharmaceutical composition may be that of a medical food, a sachet, tablet that may be crushed or dissolved in liquid. It may be in an oil, a syrup, or a paste that can be administered. Examples of oils include medium chain triglyceride (MCT) oil, vegetable oils, mineral oils or other edible oils. Formulations may include emulsifiers like lecithin of any source.

Immunotherapy utilizing IgA or IgG has demonstrated effectiveness against enteric pathogens when delivered therapeutically post-infection or concurrently with the pathogen, but have not been effective when administered prophylactically to prevent infection. There are currently no methods by which to deliver immunoglobulins for prevention of enteric pathogens. This disclosure provides a novel mechanism to anchor glycosylated secretory-bound IgA to the gut by oral co-delivery of the glycoprotein with key commensal bacteria first grown on a human milk oligosaccharide. A heterogeneous pool of human milk SIgA or a recombinant monoclonal SIgA (rSIgA) derived from a mammalian source with similar efficacy may be used. Highly specific rSIgA cocktail selective against key enteropathogens prevalent in a geographical region may be engineered and/or blended from one or more sources. Methods involve administering the cocktails to susceptible individuals and serve to protect against invading pathogens.

The individual may be a human or a non-human mammal. The non-human mammal may include, but is not limited to a pig, cow, horse, dog, cat, camel, rat, mouse, goat, sheep, or water buffalo. The non-human mammal may also include goat, sheep, water buffalo, camel or others whose milk may be consumed by humans. The non-human mammal may be an animal used in food production, a performance animal, or a domesticated pet. Any of the above may be a newborn, weaning, adult or geriatric animal. The human individual may be an infant, a preterm or premature infant who may be born with a gestational age of less than 33 weeks, the preterm babies may be a very low birth weight (VLBW), or low birth weight (LBW), a term infant (0-3 months), an infant 3-6 months, an infant (6-12 months), a weaning infant (4-12 months), a weaned infant (12 months to 2 years) and child (1-16 years), an adult (16-70 yr), or an older adult (70-100+yr). The preterm infant may be at risk of developing necrotizing enterocolitis (NEC). The infant, child or adult may be at increased risk for diarrheal diseases.

Specifically, the evidence herein demonstrates a dampening of host inflammation with or without pathogen challenge with the SIgA-commensal complex. Gut colonization or persistence of these complexes may be used to prevent or treat individuals with diseases or conditions such as inflammatory bowel disease, Crohn's disease, or other colitis, but also provide therapy for inflammatory-based diseases of the cardiovascular system (i.e. atherosclerosis), nervous system (i.e. neuropathy), immune system (autoimmunity, allergies), and metabolic system (obesity, diabetes). Or used to prevent or treat diseases that are specific to an age group, such as pre-mature infants who are highly susceptible to necrotizing enterocolitis, a disease both rooted in intestinal inflammation and pathogen exposure.

### EXAMPLE 1

### In Vitro Selection of Effective sIgA-Bacteria Complexes

Bacterial strains (Table 1) were selected based on two criteria: their ability to bind to mucin glycans (Lactobacillus species) or specific human milk oligosaccharides (HMOs) (Bifidobacterium species), and their status as a probiotic or commensal isolate. Bacteria were cultured overnight on Mann-Rogosa-Sharpe (MRS) agar, then passaged once in MRS broth anaerobically (1% inoculum) at 37° C. and passaged a second time in basal MRS (bMRS) with 1% carbohydrate. Bacteria were first tested for their growth on 1% glucose, lactose, 2′FL and

LNnT in bMRS in a 96-well plate under anaerobic conditions at 37° C. and their OD₆₀₀ measured every 30 min for 48 h to determine both their ability to grow on the carbohydrate source and their population growth curve for optimization of assays. For all further in vitro experiments, bacterial cultures were assayed during mid-log growth as determined by optical density at 600 nm, using sterile media as a reference.

TABLE 1
Growth of infant commensal Bifidobacterium species and probiotic
Lactobacillus species. Organisms did not show growth above basal media (−), grew
to between OD₆₀₀ 0.4-0.6 (+), between 0.6-1.0 (++), or above 1.0 (+++).

| | Acquired | Growth on substrate: | | | |
|---|---|---|---|---|---|
| | from: | Glucose | Lactose | 2′FL | LNnT |
| Bifidobacterium species | | | | | |
| Bifidobacterium breve SC95³⁵ (SC95) | Fecal isolate | ++ | ++ | − | ++ |
| Bifidobacterium bifidum SC555³⁶ (SC555) | Fecal isolate | ++ | − | + | − |
| Bifidobacterium longum ssp. longum | Fecal isolate | ++ | ++ | ++ | − |
| SC596³⁷ (BLL) | | | | | |
| Bifidobacterium longum ssp. infantis | Fecal isolate | ++ | ++ | + | ++ |
| ATCC 15697³⁶ (BLI) | | | | | |
| Bifidobacterium pseudocatenulatum MP80 | Fecal isolate | ++ | − | ++ | − |
| (BP) | | | | | |
| Lactobacillus species | | | | | |
| Lactobacillus acidophilus NCFM (Lacid) | probiotic | ++ | ++ | − | − |
| Lactobacillus reuteri DSM (Lreuteri) | probiotic | ++ | +++ | − | − |
| Lactobacillus rhamnosus ATCC 7469 | ATCC | ++ | + | + | − |
| (LR) | | | | | |

Binding of sIgA to bacteria. Bacteria from Table 1 were resuspended in 0.8% saline at a concentration of 1×10⁷ CFU/mL. SIgA was then added at 0, 10, 100, 500 and 1000 μg per 1×10⁷ colony forming units (CFU) of bacteria and incubated at 24° C. for 30 min. Following incubation, bacteria were pelleted at 14000×g for 4 min and washed with saline twice to remove non-adherent SIgA.

Flow cytometry and immunofluorescence: To measure binding cells were fixed using 4% w/v paraformaldehyde for 30 min, washed with PBS, then incubated with 1% bovine serum albumin (BSA) blocking buffer for 1 h. Cells were stained with 1/100 dilution SYTOT™ 9 (S34854, ThermoFisher) for live cells and goat anti-human IgA conjugated to Alexa 647 fluorophore using 1/50 dilution in 1% BSA (ab96998, Abcam). For flow cytometry, all samples were acquired and analyzed on a FACScan flow cytometer (BD Biosciences, Mountain View, Calif.) using the CellQuest software program. For live/dead analyses following in vitro digestion, cells were stained with SYTOT™ 9 (1/100 dilution) for live cells and propidium iodine (1/1000 dilution) for dead cells or cells with compromised membranes.

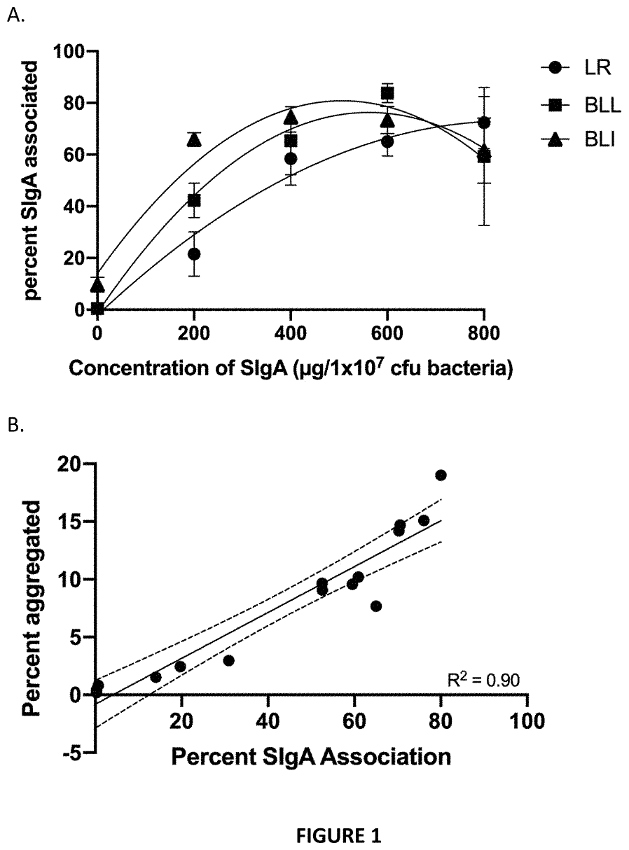

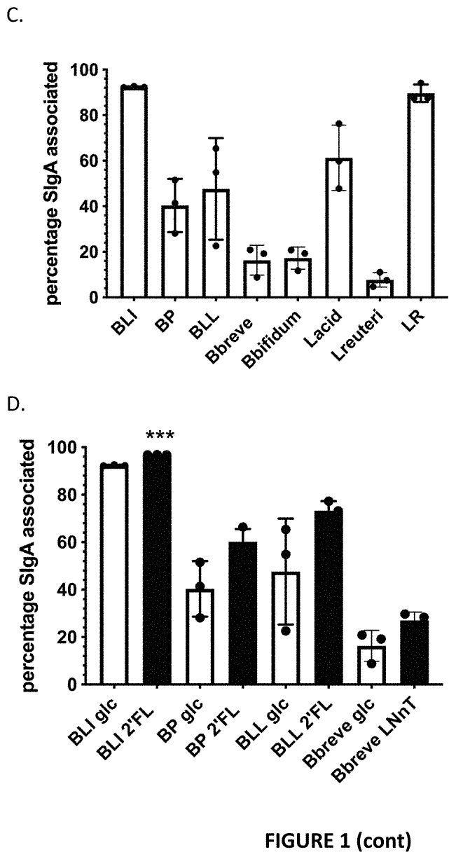

FIG. 1 depicts SIgA binding to different commensal organisms by flow cytometry. FIG. 1 Panel A demonstrates that, as an example, binding of a heterogeneous pool of SIgA to Lactobacillus rhamnosus (LR), Bifidobacterium longum subsp. longum (B. longum or BLL) and Bifidobacterium longum subsp. infantis (B. infantis or BLI) is concentration dependent. SIgA association to bacteria in these examples reaches saturation at or below concentrations found in breastmilk (n=30 per bacteria tested). FIG. 1 Panel B demonstrates aggregate formation increases upon increased association to SIgA for the commensal LR. FIG. 1 Panel C demonstrates that different bacterial strains vary in percent association with sIgA, with some showing very poor association at even the highest concentration tested (1000 μg/1e7 CFU). Bifidobacterium species tested in this experiment included B. infantis, B. longum, B. pseudocatenulatum, B. Bifidum or B. breve. Lactobacillus species tested included Lactobacillus reuteri, Lactobacillus rhamnosus, and Lactobacillus acidophilus. In FIG. 1 Panel D, Bifidobacterium species tested increased association to SIgA when first grown on 1% concentration of 2′FL in the final media preparation (BLI, p=0.0014, BP, p=0.056, and BLL, p=0.38).

In another experiment using a different ratio of SIgA and bacteria similar results were obtained. In this experiment, Bifidobacterium longum sp infantis ATCC 15697 (B. infantis), Bifidobacterium pseudocatenulatum MP80 (MP80), and Bifidobacterium breve (B. breve) were tested for their ability to bind to pooled human milk SIgA at 92.4%, 40.3%, and 16% respectively, at a concentration of 100 μg SIgA to 1e6 cfu bacteria. This binding is significantly increased when the bacteria were first grown on an HMO substrate (4.8% increase p<0.005, 61.7% increase p=0.2, and 45% increase p<0.05, respectively).

Protection from digestion using an in vitro model. To determine the extent to which the SIgA-glycan-mediated-bacterial complexes that included the Ig-bound secretory component (SC) where able to protect bacteria and SIgA from proteolytic degradation, an in vitro model was used. Simulated gastric digestion was performed by resuspending bacteria in 200 ul of 0.1% porcine pepsin (Sigma—Aldrich, St. Louis, Mo.) in PBS at pH 4. Samples were placed in an incubating shaker (New Brunswick Scientific, Edison, N.J.) at 140 rpm at 37° C. for 15 min. Cells were then pelleted and resuspended in 200 ul of 0.04% pancreatin (Sigma— Aldrich) in PBS pH 7, and the samples placed in the incubator shaker at 140 rpm at 37° C. for 5 min. Bacteria were pelleted and washed twice with PBS to remove residual enzymes, then evaluated for viability through CFU serial dilution and spot plating, and with live/dead flow cytometry analysis.

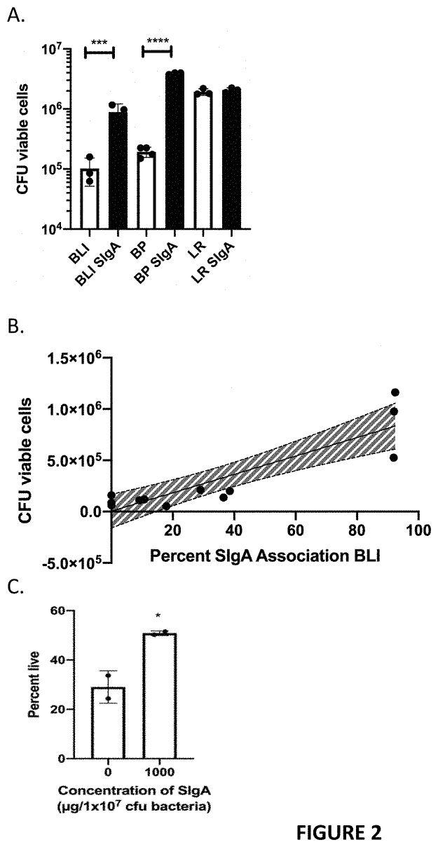

Viability of B. infantis, B. pseudocatenulatum and Lactobacillus reuteri when grown in 1% glucose was tested after being subjected to simulated gastric digestion using proteases in acidic conditions (FIG. 2). In FIG. 2 Panel A, BLI resulted in 1e5 CFU (sd 5e5 CFU) viable bacteria post-digestion when no SIgA was provided, and viability increased to 8.9e5 CFU (sd 3.3e5 CFU) when 1000 μg SIgA/1e7 CFU is provided (p=0.002). In vitro digestion resulted in 2e5 CFU viable BP bacteria when no SIgA was provided, and viability increased to 4e6 with 100 or 1000 μg SIgA/1e7 CFU wass provided (p=0.0008) and there was no significant difference in LR binding with or without SIgA. As an example, Panel B depicts a regression plot of BLI viability with SIgA association and shows a slope of 8.2e4 CFU per % population SIgA association (R²=0.53, p=0.0074). Panel C depicts a live-dead flow cytometry analysis of shows increased viable bacteria from 29.05% live (sd 6.6%) to 50.85% live (sd 0.92%) after 1000 μg SIgA/1e7 CFU is provided (p=0.04).

Pooled human milk SIgA, when complexed to HMO-grown commensals, was demonstrated to increase viability of select species after in-vitro digestion using pancreatin and porcine pepsin.

TABLE 2
SIgA protects from in vitro digestion.

| | | | Slope | | | |
|---|---|---|---|---|---|---|
| Bacteria | | | (CFU/% | | | |
| Species | Strain | Substrate | SIgA) | R² | p | S/NS |
| B. longum | 15697 | glucose | 8970 | 0.7851 | 0.0001 | S |
| spp | | 2′FL | 410.1 | 0.4561 | 0.0157 | S |
| infantis | | lactose | 76377 | 0.3038 | 0.0632 | NS |
| | | LNnT | 1214 | 0.6155 | 0.0025 | S |
| B. | MP80 | glucose | 82548 | 0.5284 | 0.0074 | S |
| pseudo- | | 2′FL | 51114 | 0.482 | 0.0122 | S |
| catenulatum | | | | | | |
| B. breve | SC95 | lactose | −106.9 | 0.02347 | 0.6345 | NS |
| | | LNnT | 223.1 | 0.2516 | 0.0966 | NS |
| L. | 7469 | glucose | −2121 | 2.03E−04 | 0.9898 | NS |
| rhamnosus | | 2′FL | −39446 | 0.1478 | 0.347 | NS |
| | | lactose | −23719 | 0.0034 | 0.8909 | NS |
| L. | NCFM | glucose | −8952 | 0.1962 | 0.1493 | NS |
| acidophilus | | lactose | 38918 | 0.3087 | 0.2523 | NS |
| L. | DSM | glucose | −38517 | 0.0438 | 0.5889 | NS |
| reuteri | 17938 | lactose | 47155 | 0.2887 | 0.2716 | NS |

In Table 2, results from the regression analysis of increased SIgA association are plotted against viable CFU counts post-digestion. Slope of the regression analysis is in viable bacteria measured in colony forming units (CFU) per percent of population associated to SIgA as measured by flow cytometry.

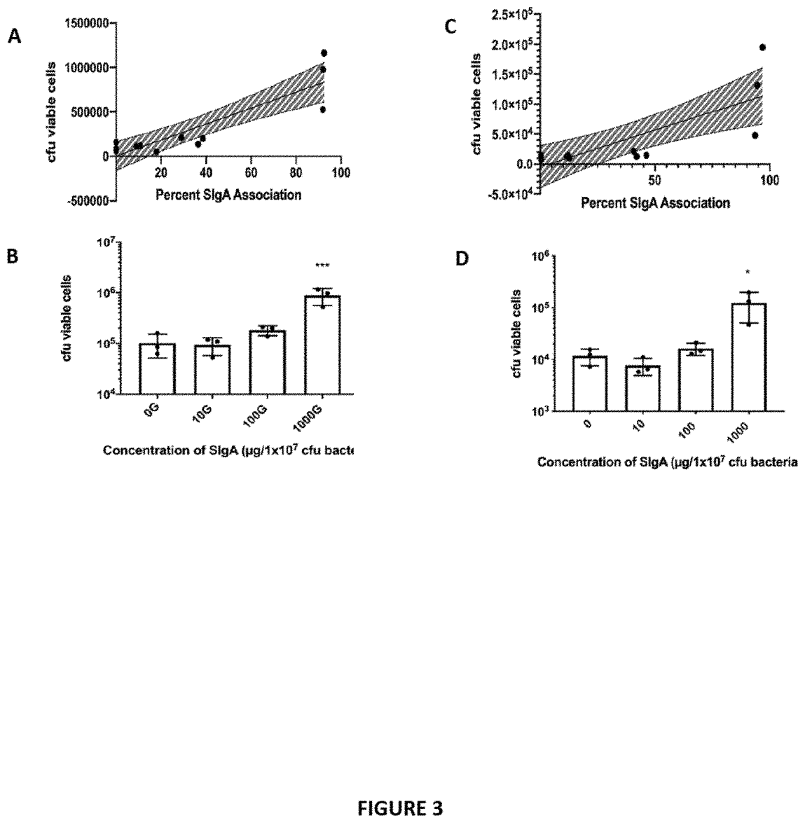

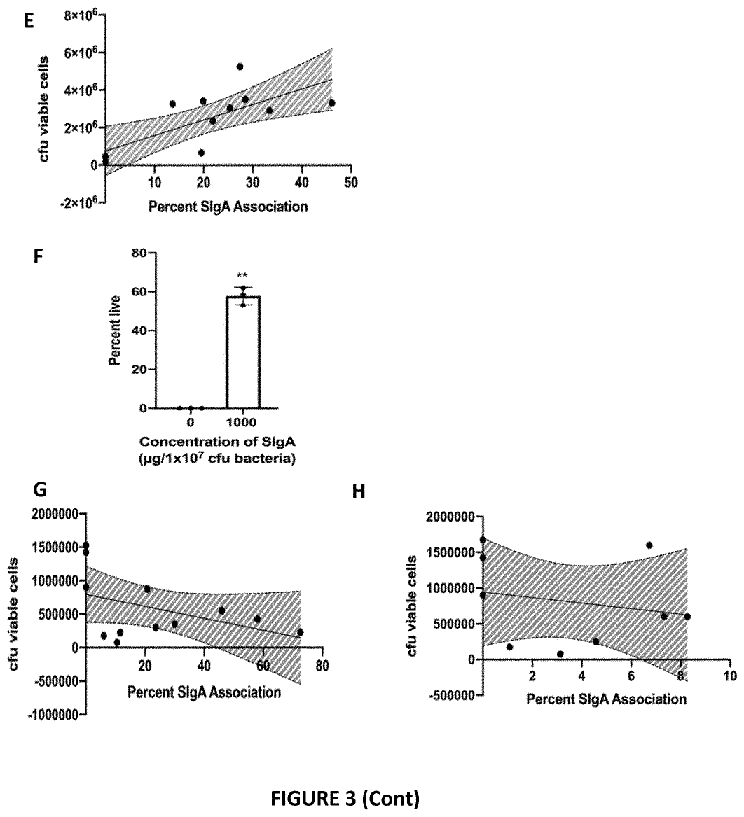

FIG. 3 demonstrates that more viable B. infantis, B. pseudocatenulatum and B. breve are recovered when it is first complexed with SIgA that were in general more susceptible to gastric digestion than the Lactobacillus. This was not true of the Lactobacillus species tested. Specifically, when grown on glucose, B. infantis shows 1e5 CFU (sd 5e5 CFU) viable bacteria post-digestion when no SIgA is provided, and viability increases to 8.9e5 CFU (sd 3.3e5 CFU) when 1000 μg SIgA/1e7 CFU is provided (p=0.002) (B), and a regression plot shows association between percent SIgA association and increased viability (R²=0.785, p=0.0001) where the p-value indicates that the slope is significantly different from zero (A). When grown on LNnT, B. infantis shows 1.2e4 viable CFU (sd 4.2e3 CFU) post-digestion when no SIgA is added, and 1.3e5 CFU (sd 7.4e4) after 1000 μg SIgA/1e7 CFU is provided (p=0.0243) (D). A regression plot shows a slope of 1.2e3 CFU per % population SIgA association (R²=0.62, p=0.0025) (C). B. pseudocatenulatum regression plot shows a slope of 8.2e4 CFU per % population SIgA association (R²=0.53, p=0.0074) (E). Live-dead analysis of B. breve shows no viable bacteria when no SIgA is added, but 57.8% (sd 4.5%) viable bacteria after 1000 μg SIgA/1e7 CFU is provided (p=0.0051). Both Lactobacillus species L. acidophilus (G) and L. reuteri (H), when grown on glucose, show a negative association to SIgA post-digestion (m=−8.9e3, R² =0.2, p=0.15 for L. acidophilus, and m=−3.9e4 CFU/% SIgA association, R²=0.044, p=0.59 for L. reuteri).

Assessment of epithelial cell adherence and barrier function. An in vitro method using colonocytes was used to assess different commensal-SIgA combinations for bacterial-epithelial cell binding, NFKB IL-8 and tight junction binding protein occludin expression.

Mammalian cell culture binding assays: Caco-2 colonic cells were co-cultured with HT29-MTX E12 cells at a ratio of 3:1 and seeded at a density of 5×10⁴ cells/well in 24-well plates, maintained as described above. On day 1 post-confluence, medium was removed, the cells washed once with PBS, and DMEM without FBS or antibiotics was added prior to binding assay. Bacteria were prepared as described above, with or without SIgA and resuspended in PBS at 1×10⁷ CFU/mL. 4×10⁵ CFU were added to the colonocytes and the plate was centrifuged at 600×g for 5 min to ensure bacteria association with the cells. After 2 h of incubation at 37° C. in 5% CO₂, medium was removed and saved for cytokine analysis. Cells were washed once with PBS, and to one set of replicates, cells were lysed with 0.5% Triton X-100 and serial dilutions of the cell suspensions were plated on MRS and incubated anaerobically at 37° C. overnight to test viability. To a second set of replicates, TRIzol (15596018; Life Technologies) was added directly to washed cells for RNA extraction.

Mammalian cell culture RT-qPCR for gene expression: Total RNA from mammalian co-culture samples were extracted via the TRIzol method. Total RNA (1 μg) was treated with Turbo DNAse (EN0521; Thermo Fisher) to remove genomic DNA, then used for reverse transcription producing cDNA, performed according to manufacturer protocol (High Capacity Complementary DNA Reverse Transcription Kit; Applied Biosystems). Gene list and primer sequences can be found in Table 2. Real-time PCR was performed with the Quantistudio 3 qPCR thermocycler (Applied Biosystems) using SyberGreen master mix (Life Technologies). Actin and GADPH were used as house-keeping genes. Analysis was performed using Quantistudio Design and Analysis Software v.1.4.3.

TABLE 3
Gene list and primer sequences for qPCR

| Gene | GeneBank # | Forward | Reverse |
|---|---|---|---|
| IL-8 | 3576 | TTTTGCCAAGGAGTGCTAAAGA | AACCCTCTGCACCCAGTTTTC |
| | | (SEQ ID NO: 1) | (SEQ ID NO: 14) |
| TNF-α | 7124 | CCTCTCTCTAATCAGCCCTCTG | GAGGACCTGGGAGTAGATGAG |
| | | (SEQ ID NO: 2) | (SEQ ID NO: 15) |
| RIPK1 | 8737 | GGGAAGGTGTCTCTGTGTTTC | CCTCGTTGTGCTCAATGCAG |
| | | (SEQ ID NO: 3) | (SEQ ID NO: 16) |
| pIgR | 5284 | AGTCCCATATTTGGTCCCGAG | AGGTGGGTGGGTAGTAGCAC |
| | | (SEQ ID NO: 4) | (SEQ ID NO: 17) |
| MUC5AC | 4586 | TGCCCCTACAACAAGAACAAC | GGAACAGCACTGGGAGTAGTT |
| | | (SEQ ID NO: 5) | (SEQ ID NO: 18) |
| MUC13 | 56667 | CAGACAGTGAGTCAACCACAAA | GGACCTGTGCTGTTTAGGGT |
| | | (SEQ ID NO: 6) | (SEQ ID NO: 19) |
| Occludin | 100506658 | ACAAGCGGTTTTATCCAGAGTC | GTCATCCACAGGCGAAGTTAAT |
| | | (SEQ ID NO: 7) | (SEQ ID NO: 20) |
| Claudin 1 | 9076 | CCTCCTGGGAGTGATAGCAAT | GGCAACTAAAATAGCCAGACCT |
| | | (SEQ ID NO: 8) | (SEQ ID NO: 21) |
| Claudin 2 | 9075 | GCCTCTGGATGGAATGTGCC | GCTACCGCCACTCTGTCTTTG |
| | | (SEQ ID NO: 9) | (SEQ ID NO: 22 |
| Claudin 6 | 9074 | TGTTCGGCTTGCTGGTCTAC | CGGGGATTAGCGTCAGGAC |
| | | (SEQ ID NO: 10) | (SEQ ID NO: 23) |
| JAM | 50848 | ATGGGGACAAAGGCGCAAG | CAATGCCAGGGAGCACAACA |
| | | (SEQ ID NO: 11) | (SEQ ID NO: 24) |
| Lysozyme | 4069 | CTTGTCCTCCTTTCTGTTACGG | CCCCTGTAGCCATCCATTCC |
| | | (SEQ ID NO: 12) | (SEQ ID NO: 25) |
| Actin | 58 | GGCATTCACGAGACCACCTAC | CGACATGACGTTGTTGGCATAC |
| | | (SEQ ID NO: 13) | (SEQ ID NO: 26) |

Brightfield microscopy: BLI and ST were cultured as described above. 1×10⁶ CFU BLI or ST were resuspended in PBS and incubated with or without 50 μg Sal4 for 30 min and then washed twice with PBS. Cells were either concentrated and smeared on a glass slide, or incubated for 30 min with at a 1:1 mix of BLI: ST, then concentrated and smeared. Smears were air-dried and heat-fixed, then stained using the Gram stain procedure. In brief, slides were saturated with crystal violet for 30 s followed by iodine for 30 s, then decolorized with 3-5 drops of acetone and rinsed with water. Finally, smears were saturated with safranin for 30 s, rinsed, and then viewed under 1000× total magnification with oil using brightfield microscopy.

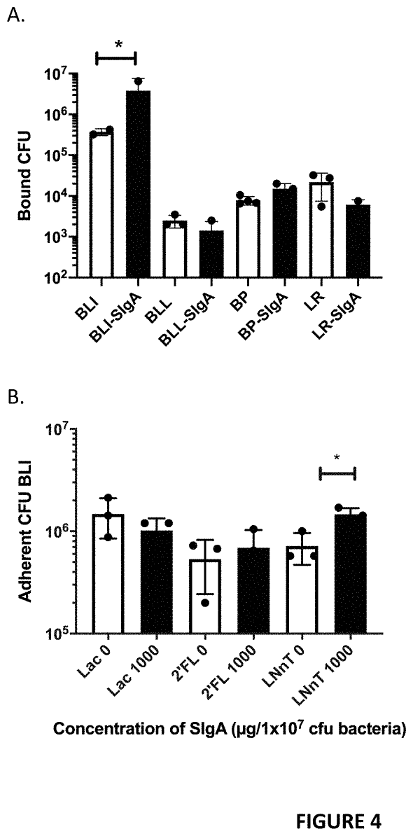

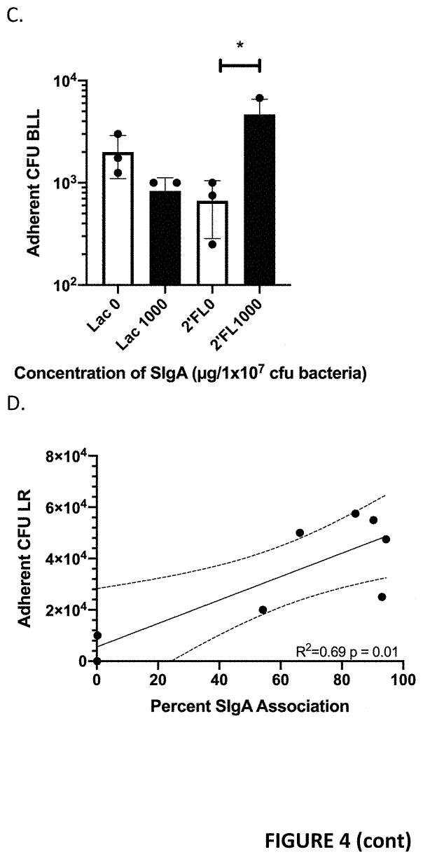

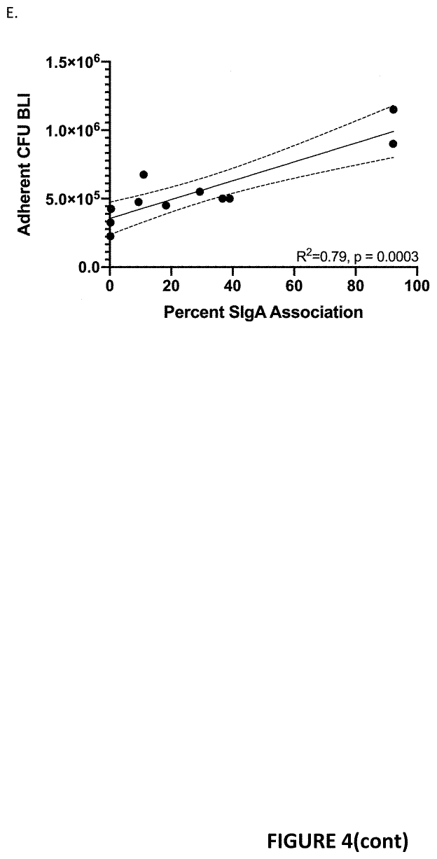

As illustrated in FIG. 4, different adherence properties can be achieved by growing or activating different bacteria with different HMO molecules or glucose. B. Longum and B. infantis to closely related species may need to be treated differently to increase adherence and effectiveness for persistence or colonization in establishing or re-establishing a niche in a microbial community In FIG. 4, BLI shows increased adherence to the co-culture with the addition of SIgA when first grown on glucose (3.5e6 CFU increased adherence, p=0.0168)(A) or LNnT (7.5e5 CFU, p=0.0164) (B), and BLL showed increased adherence to colonocytes with SIgA association only when first grown on 2′FL (4e3 increase, p=0.024) (C). A regression plot of lactose-grown LR (D) had increased adherent bacteria with increased association with SIgA (slope =37.7). BLI (E) showed similar correlation when grown on lactose (slope=4.6e3). Slope units are adherent CFU per % population associated with SIgA, and p value on regression plots indicate if the slope is not zero.

TABLE 4
Regression plot statistics showing a significant correlation between
SIgA association and increased adherence to colonocyte co-cultures
in vitro for select commensal and probiotic bacteria.

| | | | Slope | | | |
|---|---|---|---|---|---|---|
| | | | (adherent | | | |
| | | | CFU/ | | | |
| | | | % SIgA | | | S/ |
| Bacteria | Strain | Substrate | association) | R² | p | NS |
| B. infantis | 15697 | glucose | 6882.0 | 0.7868 | 0.0003 | s |
| B. infantis | 15697 | lactose | −2230.0 | 0.0401 | 0.5325 | ns |
| B. infantis | 15697 | 2'FL | 1552.0 | 0.0544 | 0.4655 | ns |
| B. infantis | 15697 | LNnT | 3606.0 | 0.2076 | 0.1366 | ns |
| B. infantis | 15697 | all (no lac) | 4887.0 | 0.2018 | 0.0068 | s |
| B. | MP80 | glucose | 14.2 | 0.0089 | 0.7720 | ns |
| pseudo- | | | | | | |
| catenulatum | | | | | | |
| B. | MP80 | 2′FL | −50.6 | 0.0939 | 0.3327 | ns |
| pseudo- | | | | | | |
| catenulatum | | | | | | |
| B. | MP80 | all | 32.0 | 0.0218 | 0.4911 | ns |
| pseudo- | | | | | | |
| catenulatum | | | | | | |
| B. longum | SC596 | glucose | 28.5 | 0.2930 | 0.0691 | NS |
| B. longum | SC596 | 2'FL | 37.7 | 0.3907 | 0.0298 | S |
| B. longum | SC596 | lactose | −13.3 | 0.1332 | 0.2434 | NS |
| B. longum | SC596 | all | 27.1 | 0.2334 | 0.0028 | S |
| B. breve | SC95 | glucose | 9083.0 | 0.2697 | 0.0836 | NS |
| B. breve | SC95 | lactose | 4617.0 | 0.6198 | 0.0069 | S |
| B. breve | SC95 | LNnT | 19516.0 | 0.2955 | 0.0677 | NS |
| B. breve | SC95 | all | 13289.0 | 0.2066 | 0.0069 | S |
| B. bifidum | SC555 | glucose | 2288.0 | 0.4013 | 0.0364 | s |
| B. bifidum | SC555 | 2'FL | 16.9 | 0.7870 | 0.0006 | s |
| B. bifidum | SC555 | all | 896.4 | 0.0486 | 0.3241 | ns |
| L. | 7469 | glucose | −138.4 | 0.2422 | 0.1041 | ns |
| rhamnosus | | | | | | |
| L. | 7469 | lactose | 456.6 | 0.6687 | 0.0131 | s |
| rhamnosus | | | | | | |
| L. | 7469 | 2'FL | 333.3 | 0.0348 | 0.6887 | ns |
| rhamnosus | | | | | | |
| L. reuteri | DSM | glucose | −9455.0 | 0.0036 | 0.8539 | ns |
| | 17938 | | | | | |
| L. reuteri | DSM | lactose | 524.1 | 0.0009 | 0.9284 | ns |
| | 17938 | | | | | |
| L. reuteri | DSM | all | −4652.0 | 0.0138 | 0.5845 | ns |
| | 17938 | | | | | |
| L. | NCFM | glucose | −3014.0 | 0.1218 | 0.2662 | ns |
| acidophilus | | | | | | |
| L. | NCFM | lactose | −3341.0 | 0.1703 | 0.1825 | ns |
| acidophilus | | | | | | |
| L. | NCFM | all | −3134.0 | 0.1371 | 0.0749 | ns |
| acidophilus | | | | | | |

Table 4, the slope is measured as adherent CFU as measured by plating after binding assays per percentage of the population associated with SIgA as measured by flow cytometry. p-value measures the significance of the slope not equal to zero.

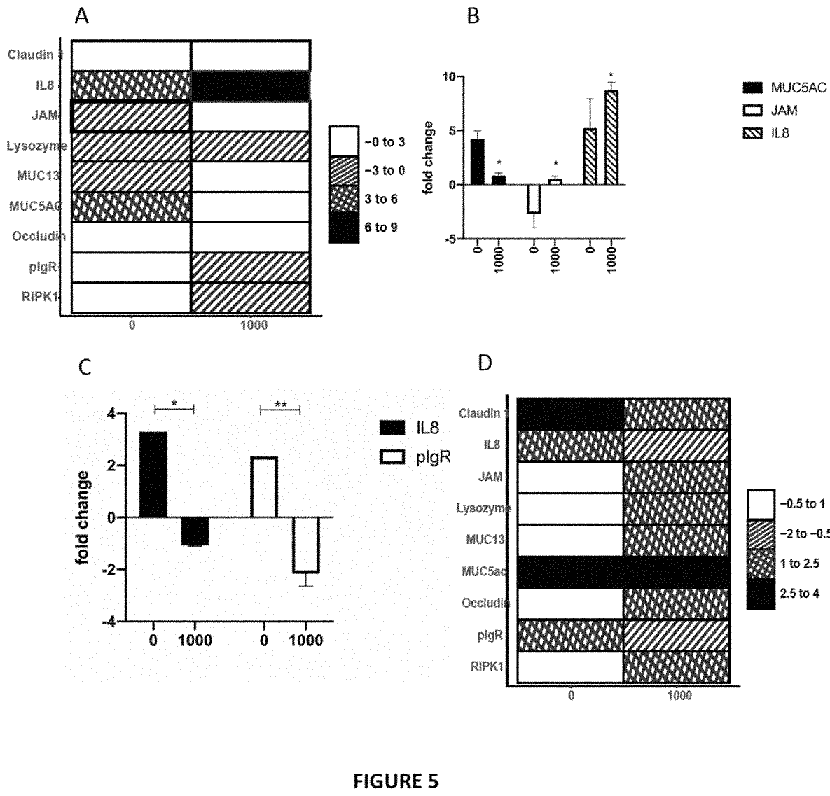

B. infantis and L. rhamnosus have differing effects on barrier function as measured by gene expression (FIG. 5). Specifically, FIG. 5 shows (A) Heat map of barrier function and immune gene expression changes to colonocytes when BLI only (0), or BLI complexed to SIgA (1000, 1000 μg SIgA per 1e7 CFU) were added, compared to PBS. (B) Fold change of IL-8 increased (p=0.0001) and barrier genes MUCSAC (p<0.05) and JAM (p<0.05) decreased when SIgA was first complexed to the BLI. (D) Heat map of gene expression changes in colonocytes when LR was added without SIgA (0) or with SIgA (1000, 1000 μg SIgA per 1e7 CFU). Gene expression for IL-8 (p<0.05) and pIgR (p<0.01) decreased 4-fold when LR was first complexed with SIgA (C).

In addition, there is a significant reduction in the neutrophil recruiting IL-8 cytokine when SIgA is first associated to the commensals prior to binding in vitro on colonocytes. This SIgA association protects these species of bacteria from proteolysis, with a greater percentage of viable bacteria remaining following in vitro digestion than when the bacteria are not associated with SIgA. SIgA association enhances the ability of B. infantis to bind to mucosal surfaces in mammalian cell culture models, and reduces the expression of the pro-inflammatory cytokine IL-8 while increasing the expression of tight junction binding proteins junctional adhesion molecule (JAM), Claudin 1 and Occludin in the mammalian colonocytes.

Protection from enteropathogens. Caco-2 cells were co-cultured with HT29-MTX E16 cells at a ratio of 3:1 and seeded at a density of 5×10⁴ cells/well in 24-well plates, maintained as described above. On day 1 post-confluence, medium was removed, the cells washed once with PBS, and DMEM with no FBS and no antibiotics was added prior to invasion assay. Sal4 is a monoclonal, polymeric IgA antibody (recombinant SIgA) that binds an immunodominant epitope within the O-antigen (O-Ag) component of lipopolysaccharide and inhibits entry of S. typhimurium into epithelial cells. 40 μL BLI with or without pre-incubation with a recombinant SIgA against Salmonella Sal4 (rSIgA) as described above, suspended in PBS at 1×10⁷ CFU/mL was added to the cells and centrifuged at 1000 rpm for 5 min to ensure bacteria association with the culture. After 1 h of incubation at 37° C., 40 μL Salmonella typhimurium (1:1 mix of JS107 and SJF10) in PBS at 1×10⁷ CFU/mL was added to the cells and centrifuged as described for 1 h incubation at 37° C. Alternately, 40 μL PBS was added alone or with 1.2 μg Sal4 for 2 h as controls. After final incubation, medium was removed and saved at −80° C. for cytokine analysis. Cells were washed once with 1×PBS, and to one set of replicates, gentamycin was added at 150 μg/mL for 45 min to kill extracellular bacteria and then washed twice with PBS. A second set of replicates was evaluated for total adhered and invaded bacteria. All cells were lysed with 0.5% Triton X-100 and serial dilutions of the cell suspensions plated on MRS anaerobically and blue/white screening agar with kanamycin aerobically at 37° C. overnight. To a third set of replicates, TRIzol was added directly to washed cells for RNA extraction.

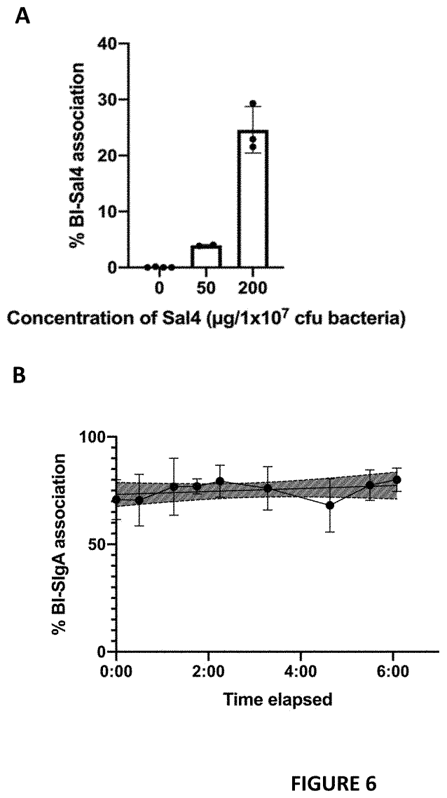

FIG. 6 highlights the concentration dependence and stability of BLI-Sal4. In FIG. 6, SIgA association with BLI is concentration dependent (A) and stable over a 6 hour time interval (B). L. reuteri association with SIgA shows a loss of over 7% SIgA-bacteria complexes after deglycosylation. In some cases, fecal bacteria coated in SIgA demonstrated a loss of association between bacteria and SIgA of over 12% when the complex is treated with either PNGase F, or EndoBI-1 (an endoglycosidase from B. infantis that cleaves N-glycan).

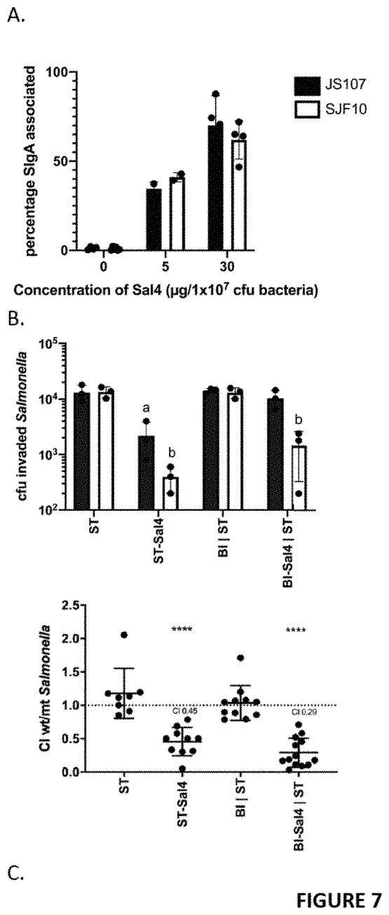

FIG. 7. Sal4 association to both strains of Salmonella was concentration-dependent (A) and although at 5 μg Sal4 per 1×10⁷ CFU both strains has similar association to Sal4, at 30 μg Sal4 there was higher association of the wild-type JS107 than the mutant SJF10 to the antibody. Sal4 prevented invasion of both the wild-type (white columns) and the mutant (black columns) into colonocytes (B) when pre-incubated (ST-Sal4), but when the BLI-Sal4 complex was added to the colonocytes prior to Salmonella enterica serovar Typhimurium (ST) challenge, only the wild-type was prevented from invasion (BLI-Sal4 ST). A competitive index calculation (C) of all invasion assays (n=9 from three separate trials) shows a selective reduction of the wild-type strain both when Sal4 was added directly to the ST mix prior to colonocyte challenge (ST-Sal4) or when BLI-Sal4 complex was added first to the colonocytes prior to ST challenge (BLI-Sal4|ST).

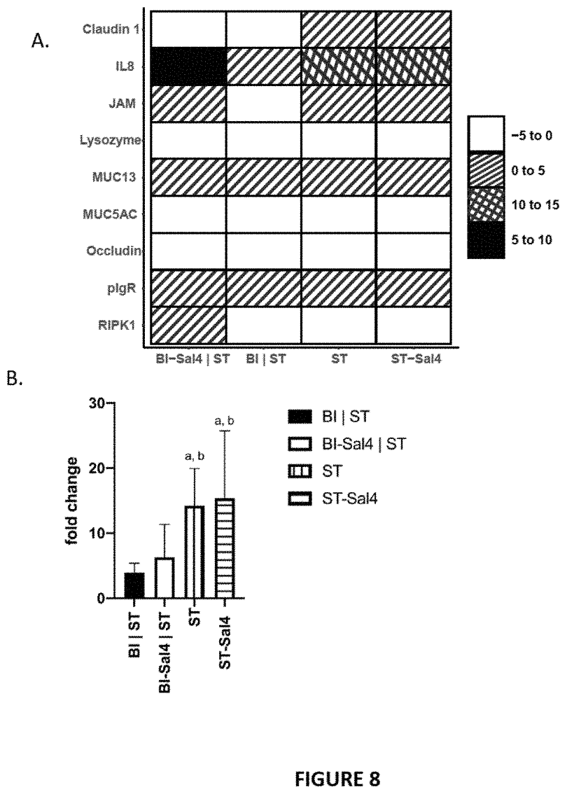

In FIG. 8, Gene expression changes in colonocytes as compared to PBS control. (A) A heatmap of barrier function genes including MUCSAC (mucin protein produced by HT-29 cells), MUC13 (mucin protein produced by Caco2 cells), Claudin 1, Occludin and Junction Adhesion Molecule (JAM), and immune function genes including interleukin 8 (IL8), lysozyme , polymeric Ig receptor (pIgR), and Receptor Interacting Protein Kinase 1 (RIPK1) for cells challenged with BI-ST (B. infantis alone), BI-Sal4-ST, ST or ST-Sal4). (B) IL-8 gene expression changes with various treatments over PBS-treated colonocytes.

When human colonocytes were first treated with a SIgA-B. infantis complex and then challenged with Salmonella enterica serovar Typhimurium, the number of invading pathogen was reduced by 94% (p<0.05), and the pro-inflammatory cytokine IL-8 was reduced by over 50% (p <0.05).

FIG. 9: Brightfield microscopy of a Gram stain of various combinations of BLI, Sal4 and ST. (A) BLI with no Sal4 has natural clustering, but is increased in aggregation when 200 μg per 1×10⁷ CFU was added (D). ST shows no aggregate formation without Sal4 (B) but has a high degree of aggregation with 30 μg Sal4 per 1×10⁷ CFU (E). BLI and ST together show little association without Sal4 (C), but have significant BLI-ST clustering when BLI is first pre-incubated with Sal4 for 30 m followed by the addition of ST (F).

### EXAMPLE 2

### In Vivo Selection for Improved Colonization

Finally, this SIgA association improves enteric colonization of B. infantis in BALBc mice, which is further enhanced when mice are supplemented with HMO.

Mice: 6-week old female BALBc mice were purchased from The Jackson Labs. Mice were housed in an American Association for the Accreditation of Laboratory Animal Care-accredited facility, and procedures were conducted in compliance with the University of California Institutional Animal Care and Use Committee. Mice were co-housed 5 mice per cage in the Training and Research Animal Care Services vivarium at the University of California, Davis under conventional conditions with free access to standard chow RM1P (801151, Special Diet Services, Witham, England) and sterilized tap water with or without 2′FL. Mice were acclimated for 1 week prior to treatment. Mice were euthanized by decapitation following deep anesthesia with 100 mg/kg ketamine and 10 mg/kg xylazine administered by intraperitoneal injection.

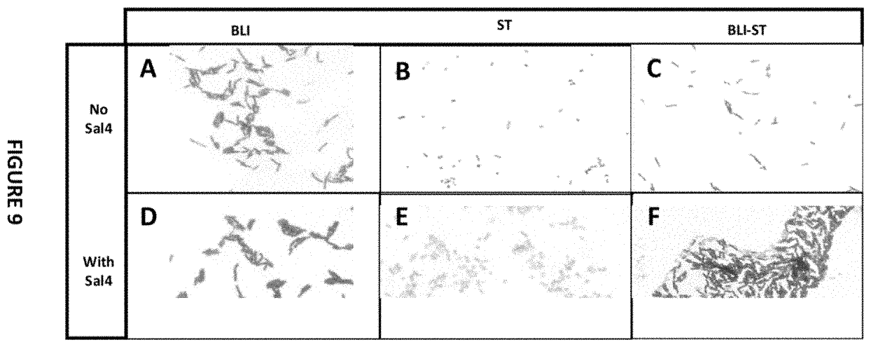

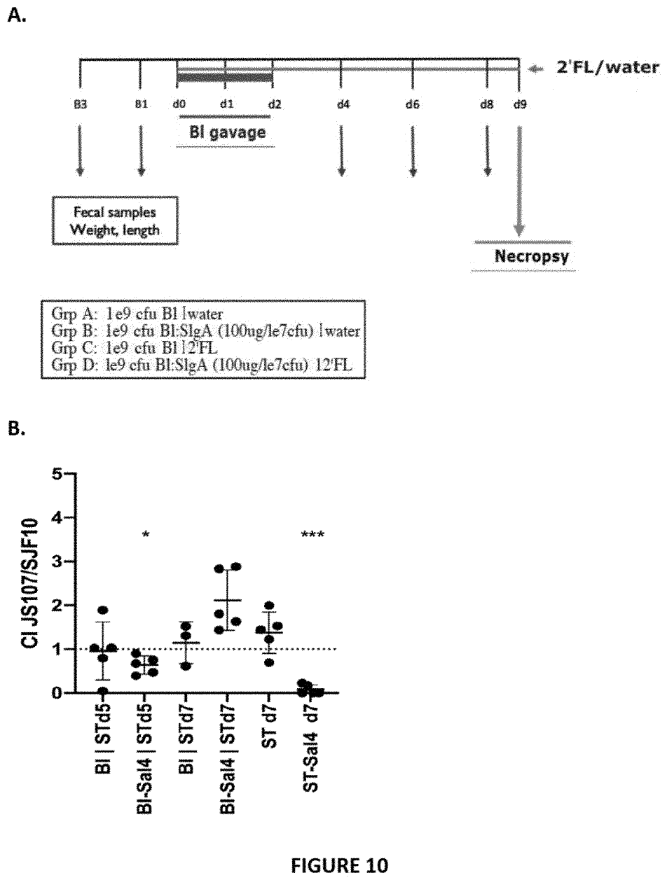

The schematic for the mouse experiments is outlined in FIG. 10A. Briefly, BI, BI-Sal4 complex, or PBS was provided via oral-gastric gavage to 7 week-old female BALBc mice for three days, followed by Salmonella enterica serovar Typhimurium (ST) challenge on either d5 or d7. Mice were provided 10% 2′FL in their drinking water for the trial. Competitive index (B) shows the ratio of wild-type JS107 to mutant SJF10 strains collected from the Peyer's patches of mice during necropsy. BI-Sal4 complex reduced wild-type JS107 by roughly 30% over mutant SJF10 when ST was challenged 3 days after oral administration of the probiotic complex (d5), but there was no effect when ST was challenged two days later (d7). Student t-test of the CI of each treatment vs untreated ST control (ST d7) revealed statistical significance of both ST-Sal4 positive control (CI=0.25, p=0.0001) and BI-Sal4 STd5 (CI =0.64, p <0.01).

Tissue Collection: Fecal samples were collected aseptically every 2 days for BLI detection. During necropsy, segments of duodenum, ileum, and colon were sectioned. First, Peyer's patches (PP) were collected and stored in 200 μL PBS in bead-beating tubes for homogenization and plating on blue/white screening agar. Duodenum, ileum, colon and cecum contents were collected into sterile tubes for bacterial analysis.

Bacterial plating and counting: PP, luminal contents and fecal samples were subjected to bead beating for 60 s at 4 m/s using a FastPrep homogenizer and 2 mm zirconium ceramic beads. Homogenate was serially diluted and plated on LB agar with kanamycin, x-gal and IPTG (blue-white screening agar) for differentiating JS107 and SJF10 strains, or further processed for DNA extraction and RT-qPCR for quantitation of BLI.

Microbial DNA extraction: DNA was extracted from homogenized duodenum, ileum, cecum, and colon contents and mouse fecal samples using the KingFisher Flex instrument and the Quick-DNA Fecal/Soil Microbe 96 Magbead Kit (Zymo Research D6011-FM) as per manufacturer's protocol. Briefly, samples were homogenized in 700 μL Lysis Solution in bead-bashing tubes containing 0.1 and 0.5 mm ceramic beads at 4 m/s for 3 min using a FastPrep homogenizer. 200 μL of homogenate was transferred to a deep-well block 96-well plate containing 600 μL Quick-DNA MagBinding Buffer and 25 μL MagBinding Beads. Samples were mixed for 10 min, then the magnet was engaged and samples transferred to a pre-wash buffer and shaken for 5 min. The magnet was engaged and samples were transferred to gDNA Wash Buffer twice, mixing 5 min each time. Finally, the magnet was engaged and samples were transferred to and elution plate with 50 μL DNA Elution Buffer.

Strain-Specific qPCR: BLI was detected in fecal samples and intestinal contents following DNA extraction as described in Frese et al.¹⁵ using the following primers: BLON0915F 5′CGTATTGGCTTTGTACGCATTT3′ and BLON0915R 5′ ATCGTGCCGGTGAGATTTAC3′. RT-qPCR reaction mixture and thermocycling conditions were consistent with those for gene expression analysis.

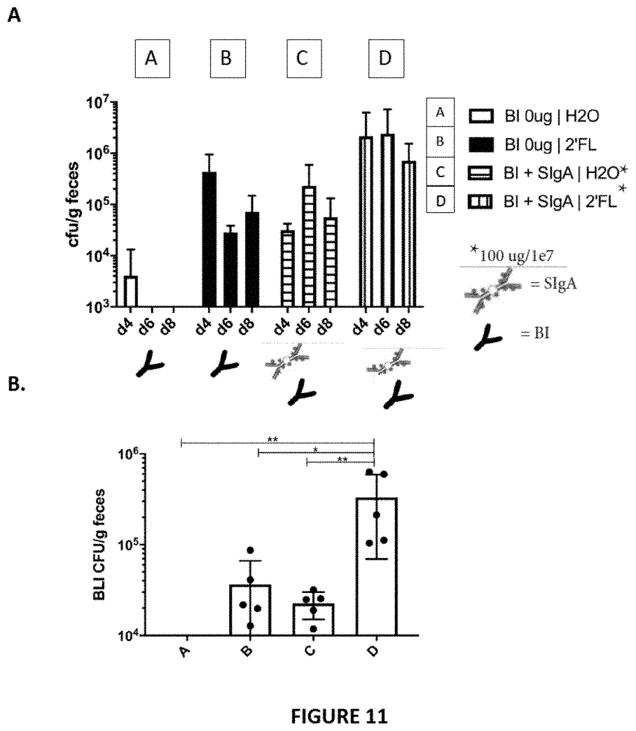

FIG. 11. (A) BLI persistence as detected by CFU/g feces in BALBc mice 1 day (d4), 3 days (d6) and 5 days (d8) post-oral administration. (B) Persistence data only for 5 days post-gavage (d8). Treatment groups were as follows: A: BLI only with no Sal4 and mice provided water. B: BLI with no Sal4 and mice provided 10% 2′FL. C: BLI pre-incubated with 100 μg per 1e7 CFU, and mice provided water. D: BLI pre-incubated with 100 μg per 1e7 CFU, and mice provided 2′FL. Data shows that 2′FL alone is sufficient to improve persistence (A to B), that Sal4 alone can improve persistence (A to C), and that there is a combination effect (D) of both Sal4 and 2′FL.

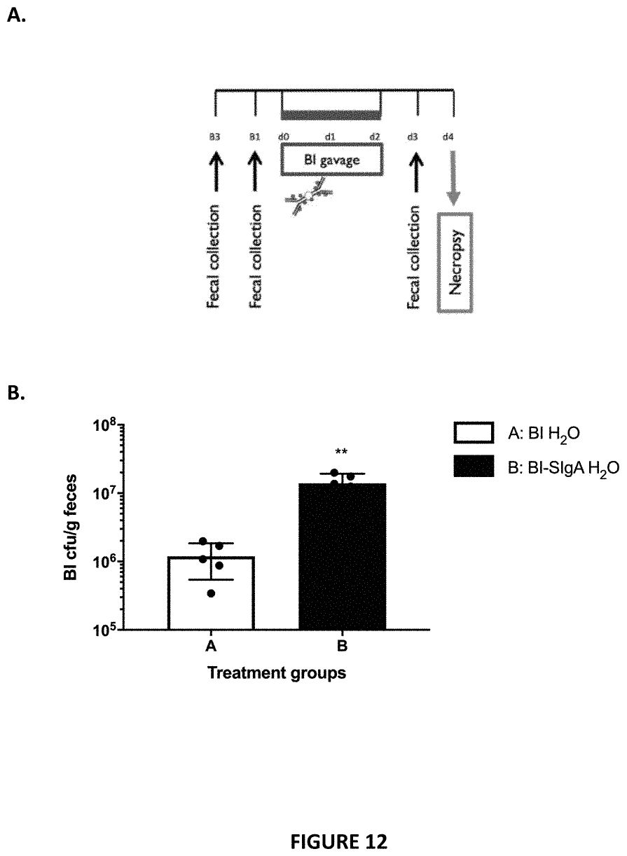

A second experimental design is highlighted in FIGS. 12 and 12 B depicts that when B. infantis is pre-incubated with milk SIgA, B. infantis is recovered 10× higher concentration in the feces of BALBc mice one day post oral administration compared to those not pre-incubated with milk SIgA, indicating improved protection from digestion.

This SIgA association increased the colonization of B. infantis in the gastrointestinal tract of 5-6 week old BALBc mice by a log-scale of 4 (p<0.1) when mice were fed water and a log-scale of 5 (p=0.1) when mice were supplemented with an HMO (10% 2′ fucosyllactose).

In addition to improved colonization, SIgA complexed to the commensal B. infantis has also shown to protect against enteropathogenic infection in vitro and in vivo. In 5-6 week old BALBc mice, provision of a SIgA-BI complex followed by Salmonella infection 3-days post-supplementation decreased invasion of the pathogen at the same level as the pre-incubation of the immunoglobulin with the pathogen (p<0.05). These data confirm the utility of a SIgA-activated commensal complex on the modification of intestinal microbial communities and in the prevention of enteric infection.

In summary, a significant reduction in enteropathogenic invasion both in vitro in human colonocytes, and in vivo in BALBc mice, was observed when SIgA-associated commensals are introduced to the cells or animal prior to pathogen challenge, but not when either the commensal or SIgA alone are provided by a glycan-mediated mechanism that may be enhanced with activated bacteria to prime them for stably adhering to the immunoglobulin.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

What is claimed is:

1. A method of stimulating Bifidobacterium persistence or viability in the intestinal microbiome of an individual, the method comprising, administering a composition comprising a Bifidobacterium and a glycosylated immunoglobulin to the individual.

2. The method of claim 1, wherein the glycosylated immunoglobulin is selected from the group consisting of secretory immunoglobulin A (SIgA), dimeric IgA (dIgA), monomeric IgA, secretory IgM (SIgM), IgM, IgG, IgE, IgD and a glycosylated immunoglobulin fragment thereof.

3. The method of claim 2, wherein the glycosylated immunoglobulin is secretory IgA (SIgA).

4. The method of claim 2 wherein the immunoglobulin fragment is a glycopeptide or glycoprotein comprising at least 10, 20, 40, 60, 80, or at least 100 amino acids.

5. The method of claim 2, wherein the composition of glycosylated immunoglobulin, or fragment thereof, has affinity for one or more specific enteric pathogen or toxin of viral, fungal, or bacterial origin.

6. The method of claim 5, wherein the specific enteric pathogen or enteric toxin composition of immunoglobulins, or fragments thereof, is selected from rotavirus, Salmonella, Shigella, Camplyobacter, Cryptosporidium, Escherichia coli, Clostridium difficile, Clostridium enterotoxin from Clostridium perfringens, Cholera toxin from Vibrio cholerae, Staphylococcus enterotoxin B from Staphylococcus aureus, Shiga toxin from Shigella dysenteriae, those from Bacillus cereus, and Toxin A or B from Clostridium difficile.

7. The method of claim 6, wherein the immunoglobulin, or fragment thereof, is recombinant or otherwise synthetically derived.

8. The method of claim 6, wherein the immunoglobulin composition is a heterogeneous milk-derived immunoglobulin fraction.

9. The method of claim 8, wherein the glycosylated immunoglobulin is not from the mother of the individual.

10. The method of any of the above claims, wherein the immunoglobulin and the Bifidobacterium form an immunoglobulin-Bifidobacterium complex prior to administration.

11. The method of claim 10, wherein the immunoglobulin-Bifidobacterium complex is formed through interaction between aglycan portion of the immunoglobulin, or fragment thereof, and surface glycans of the Bifidobacterium.

12. The method of any of the above claims, wherein the composition of Bifidobacterium and immunoglobulin are components of a food product or a pharmaceutical composition.

13. The method of claim 12, wherein the food product is selected from the group consisting of infant formula, follow-on formula, toddler's beverage, milk, soy milk, fermented milk, fruit juice, fruit-based drinks, post-surgery recovery drink, meal replacers, and sports drink.

14. The method of claim 13, wherein the food product is a powder.

15. The method of claim 10, wherein the Bifidobacterium is selected from the group consisting of B. longum subsp. infantis, B. longum subsp. longum, B. pseudocatenulatum, B. bifidum, B. kashiwanohense, B. adolescentis, and B. breve.

16. The method of claim 10, wherein the Bifidobacterium is B. longum subsp. infantis.

17. The method of claim 10, wherein the B. longum subsp. infantis is activated.

18. The method of claim 17, wherein the composition comprises an activated B. infantis is prepared by activating with a human milk oligosaccharide (HMO) during fermentation.

19. The method of claim 18, wherein the HMO for activating is selected from at least one of lacto-N-biose (LNB), N-acetyl lactosamine, lacto-N-triose, lacto-N-tetraose (LNT), lacto-N-neotetraose (LNnT), fucosyllactose (FL), lacto-N-fucopentaose (LNFP), lactodifucotetraose, (LDFT) sialyllactose (SL), disialyllacto-N-tetraose (DSLNT), 2′-fucosyllactose (2FL), 3′-sialyllactosamine (3SLN), 3′-fucosyllactose (3FL), 3′-sialyl-3-fucosyllactose(3S3FL), 3′-sialyllactose (3SL), 6′-sialyllactosamine (6SLN), 6′-sialyllactose (6SL), difucosyllactose (DFL), lacto-N-fucopentaose I (LNFPI), lacto-N-fucopentaose II (LNFPII), lacto-N-fucopentaose III (LNFPIII), lacto-N-fucopentaose V (LNFPV), sialyllacto-N-tetraose (SLNT), and their derivatives.

20. The method of any of the above claims, further comprising administering one or more polysaccharide to the individual in a sufficient amount to enhance colonization of the gut by the Bifidobacterium compared to not administering the polysaccharide.

21. The method of any of the above claims, further comprising administering one or more oligosaccharide to the individual in a sufficient amount to enhance colonization of the gut by the Bifidobacterium compared to not administering the oligosaccharide.

22. The method of claim 21, wherein the oligosaccharide is a human milk oligosaccharide is selected from one or more of lacto-N-biose (LNB), N-acetyl lactosamine, lacto-N-triose, lacto-N-tetraose (LNT), lacto-N-neotetraose (LNnT), fucosyllactose (FL), lacto-N-fucopentaose (LNFP), lactodifucotetraose, (LDFT) sialyllactose (SL), disialyllacto-N-tetraose (DSLNT), 2′-fucosyllactose (2FL), 3′-sialyllactosamine (3SLN), 3′-fucosyllactose (3FL), 3′-sialyl-3-fucosyllactose(3S3FL), 3′-sialyllactose (3SL), 6′-sialyllactosamine (6SLN), 6′-sialyllactose (6SL), difucosyllactose (DFL), lacto-N-fucopentaose I (LNFPI), lacto-N-fucopentaose II (LNFPII), lacto-N-fucopentaose III (LNFPIII), lacto-N-fucopentaose V (LNFPV), sialyllacto-N-tetraose (SLNT), their derivatives.

23. The method of any of the above claims, wherein the composition further comprises a Lactobacillus, wherein the Lactobacillus is selected from the group consisting of L. acidophilus, L. rhamnosus, L. casei, L. paracasei, L. plantarum and L. reuteri.

24. The method of claim 23, wherein the Lactobacillus is L. rhamnosus or L. reuteri.

25. A pharmaceutical composition or food product comprising Bifidobacterium and an immunoglobulin in a dose sufficient to enhance colonization of the Bifidobacterium compared to administration of the Bifidobacterium alone.

26. A composition of 25, wherein the immunoglobulin is glycosylated.

27. The pharmaceutical composition or food product of claim 26, wherein the immunoglobulin is selected from the group consisting of secretory immunoglobulin A (SIgA), IgA, IgM, IgG, IgE, IgD and a glycosylated immunoglobulin fragment thereof.

28. The pharmaceutical composition or food product of claim 27, wherein the immunoglobulin fragment is a glycopeptide or glycoprotein comprising at least 10, 20, 40, 60, 80, or 100 amino acids.

29. The pharmaceutical composition or food product of claim 28, wherein the food product is selected from the group consisting of human milk product, human milk fortifiers (bovine or human), processed donor milk, or milk fractions, infant formula, follow-on formula, toddler's beverage, milk, soy milk, fermented milk, fruit juice, fruit-based drinks, meal replacer, and sports drink.

30. The food product of any one of claims 25-29, wherein the food product comprises a powder.

31. The pharmaceutical composition or food product of claim 25, wherein the Bifidobacterium is selected from the group consisting of B. longum subsp. infantis, B. longum subsp. longum, B. pseudocatenulatum, B. bifidum, B. kashiwanohense, B. adolescentis, and B. breve.

32. The pharmaceutical composition or food product of claim 31 wherein the Bifidobacterium comprises B. longum subsp. infantis.

33. The pharmaceutical composition or food product of claim 32, wherein the B. longum subsp. infantis is activated.

34. The pharmaceutical composition or food product of claim 25, further comprising one or more polysaccharide that enhances colonization by the Bifidobacterium of the gut of an individual receiving the pharmaceutical composition or food.

35. The pharmaceutical composition or food product of claim 25, further comprising one or more oligosaccharide that enhances colonization by the Bifidobacterium of the gut of an individual receiving the pharmaceutical composition or food.

36. The pharmaceutical composition or food product of claim 25, further comprising a human milk oligosaccharide.

37. The pharmaceutical composition or food product of claim 25 wherein the form of such pharmaceutical composition or food product comprises a capsule, tablet, oil suspension, or sachet.

38. The pharmaceutical composition or food product of claim 25 wherein such pharmaceutical composition or food product is in a dried form.

39. The method of administering the composition or performing the method of any preceding claim, wherein administration is performed to treat or prevent a condition or disease.

40. The method of claim 39, wherein the condition or disease is dysbiosis, colic, diaper rash an inflammatory disease of the intestine, cardiovascular or nervous system, an auto-immune disease, a metabolic disease or an infection.

41. The method of claim 40, wherein the infection is caused by an enteric pathogen.

42. The method of any of the above claims, wherein the individual is a human or non-human mammal.

43. The method of claim 10 wherein the non-human mammal is selected from the group consisting of pig, cow, horse, dog, cat, camel, rat, mouse, goat, sheep, and water buffalo.

44. The method of claim 42 wherein the human is a preterm infant, a term infant, a child, adult or older adult.

45. A method of making an immunoglobulin-bacteria complex, comprising
a. activating a commensal organism;
b. selecting one or more SIgA; and
c. combining (a) and (b).


SEQUENCE LISTING (28 sequences)

SEQ ID NO: 1 - DNA, 22 bp, Artificial Sequence
  Description of Artificial Sequence Synthetic primer
ttttgccaaggagtgctaaaga

SEQ ID NO: 2 - DNA, 22 bp, Artificial Sequence
  Description of Artificial Sequence Synthetic primer
cctctctctaatcagccctctg

SEQ ID NO: 3 - DNA, 21 bp, Artificial Sequence
  Description of Artificial Sequence Synthetic primer
gggaaggtgtctctgtgtttc

SEQ ID NO: 4 - DNA, 21 bp, Artificial Sequence
  Description of Artificial Sequence Synthetic primer
agtcccatatttggtcccgag

SEQ ID NO: 5 - DNA, 21 bp, Artificial Sequence
  Description of Artificial Sequence Synthetic primer
tgcccctacaacaagaacaac

SEQ ID NO: 6 - DNA, 22 bp, Artificial Sequence
  Description of Artificial Sequence Synthetic primer
cagacagtgagtcaaccacaaa

SEQ ID NO: 7 - DNA, 22 bp, Artificial Sequence
  Description of Artificial Sequence Synthetic primer
acaagcggttttatccagagtc

SEQ ID NO: 8 - DNA, 21 bp, Artificial Sequence
  Description of Artificial Sequence Synthetic primer
cctcctgggagtgatagcaat

SEQ ID NO: 9 - DNA, 20 bp, Artificial Sequence
  Description of Artificial Sequence Synthetic primer
gcctctggatggaatgtgcc

SEQ ID NO: 10 - DNA, 20 bp, Artificial Sequence
  Description of Artificial Sequence Synthetic primer
tgttcggcttgctggtctac

SEQ ID NO: 11 - DNA, 19 bp, Artificial Sequence
  Description of Artificial Sequence Synthetic primer
atggggacaaaggcgcaag

SEQ ID NO: 12 - DNA, 22 bp, Artificial Sequence
  Description of Artificial Sequence Synthetic primer
cttgtcctcctttctgttacgg

SEQ ID NO: 13 - DNA, 21 bp, Artificial Sequence
  Description of Artificial Sequence Synthetic primer
ggcattcacgagaccacctac

SEQ ID NO: 14 - DNA, 21 bp, Artificial Sequence
  Description of Artificial Sequence Synthetic primer
aaccctctgcacccagttttc

SEQ ID NO: 15 - DNA, 21 bp, Artificial Sequence
  Description of Artificial Sequence Synthetic primer
gaggacctgggagtagatgag

SEQ ID NO: 16 - DNA, 20 bp, Artificial Sequence
  Description of Artificial Sequence Synthetic primer
cctcgttgtgctcaatgcag

SEQ ID NO: 17 - DNA, 20 bp, Artificial Sequence
  Description of Artificial Sequence Synthetic primer
aggtgggtgggtagtagcac

SEQ ID NO: 18 - DNA, 21 bp, Artificial Sequence
  Description of Artificial Sequence Synthetic primer
ggaacagcactgggagtagtt

SEQ ID NO: 19 - DNA, 20 bp, Artificial Sequence
  Description of Artificial Sequence Synthetic primer
ggacctgtgctgtttagggt

SEQ ID NO: 20 - DNA, 22 bp, Artificial Sequence
  Description of Artificial Sequence Synthetic primer
gtcatccacaggcgaagttaat

SEQ ID NO: 21 - DNA, 22 bp, Artificial Sequence
  Description of Artificial Sequence Synthetic primer
ggcaactaaaatagccagacct

SEQ ID NO: 22 - DNA, 21 bp, Artificial Sequence
  Description of Artificial Sequence Synthetic primer
gctaccgccactctgtctttg

SEQ ID NO: 23 - DNA, 19 bp, Artificial Sequence
  Description of Artificial Sequence Synthetic primer
cggggattagcgtcaggac

SEQ ID NO: 24 - DNA, 20 bp, Artificial Sequence
  Description of Artificial Sequence Synthetic primer
caatgccagggagcacaaca

SEQ ID NO: 25 - DNA, 20 bp, Artificial Sequence
  Description of Artificial Sequence Synthetic primer
cccctgtagccatccattcc

SEQ ID NO: 26 - DNA, 22 bp, Artificial Sequence
  Description of Artificial Sequence Synthetic primer
cgacatgacgttgttggcatac

SEQ ID NO: 27 - DNA, 22 bp, Artificial Sequence
  Description of Artificial Sequence Synthetic primer
cgtattggctttgtacgcattt

SEQ ID NO: 28 - DNA, 20 bp, Artificial Sequence
  Description of Artificial Sequence Synthetic primer
atcgtgccggtgagatttac